(19) United States
(12) Patent Application Publication
(10) Pub. No.: US 2014/0256664 A1
(43) Pub. Date: Sep. 11, 2014

(54) COMPOSITIONS AND METHODS FOR A MYCOBACTERIUM TUBERCULOSIS DRUG SUSCEPTIBILITY TEST

(71) Applicants: Eric R. Houpt, Keswick, VA (US); Kimberly A. Kelly, Crozet, VA (US); Suporn Pholwat, Charlottesville, VA (US)

(72) Inventors: Eric R. Houpt, Keswick, VA (US); Kimberly A. Kelly, Crozet, VA (US); Suporn Pholwat, Charlottesville, VA (US)

(73) Assignee: University of Virginia Patent Foundation, Charlottesville, VA (US)

(21) Appl. No.: 14/117,472

(22) PCT Filed: May 11, 2012
(86) PCT No.: PCT/US12/37472

§ 371 Date: Apr. 25, 2014

Related U.S. Application Data
(60) Provisional application No. 61/485,676, filed on May 13, 2011.

(51) Int. Cl. C12Q 1/68; A61K 31/506; A61K 31/7036; A61K 31/133; A61K 31/4409; A61K 31/496

(52) U.S. Cl. CPC C12Q 1/686; A61K 31/4409; A61K 31/496; A61K 31/7036; A61K 31/133; A61K 31/506
USPC 514/37; 514/354; 514/254.11; 514/669; 514/40; 514/41; 514/256; 435/6.12

(57) ABSTRACT

The present application discloses rapid Mycobacterium tuberculosis drug susceptibility utilizing real-time PCR of mycobacteriophage D29 DNA. One protocol involves culturing Tb isolates for 48 hours with and without drugs at critical concentrations, followed by incubation with 103 pfu/ml of D29 mycobacteriophage for 24 hours and then real-time PCR. Many drugs can be incubated instantly with Tb and phage. The change in phage DNA real-time PCR cycle threshold (Ct) between control Tb and Tb treated with drugs was calculated and correlated with conventional agar proportion drug susceptibility results. Specifically, 9 susceptible clinical isolates, 22 MDR, and 1 XDR Tb strains were used and Ct control−Ct drug cutoffs of between +0.3 and −6.0 yielded 422/429 (98%) accurate results for the drugs tested. The Ct values correlated with isolate minimal inhibitory concentration (MIC) for most agents. This D29 qPCR assay offers a rapid, accurate, 1-3 day phenotypic drug susceptibility test.

### CROSS REFERENCE TO RELATED APPLICATIONS

This application is entitled to priority pursuant to 35 U.S.C. §119(e) to U.S. provisional patent application No. 61/485,676, filed on May 13, 2011.

### STATEMENT REGARDING FEDERALLY SPONSORED RESEARCH OR DEVELOPMENT

This invention was made with government support under Grant No. R01 AI093358 awarded by The National Institutes of Health. The government has certain rights in the invention.

### BACKGROUND

Tuberculosis (TB) is one of the leading causes of morbidity and mortality worldwide and in particular in HIV positive individuals. There have been few new antimicrobials developed for the treatment of TB and resistance to first line therapeutics isoniazide (NH) and rifampicin (Rif) are common. Strains displaying multi (MDR) and extended (XDR) drug resistance have become a major problem in developing countries.

Multidrug resistant tuberculosis, defined as resistance to isoniazid and rifampin, occurred in an estimated 440,000 individuals worldwide in 2008 (28). Cure rates are dismal compared to drug-susceptible tuberculosis (12). Determination of appropriate therapy requires knowledge of the drug resistance profile of the organism. Molecular assays for common resistance mutations to isoniazid and rifampin are of great value yet once these have been identified the challenge arises of identifying a regimen of several medications to which the M. tuberculosis isolate is susceptible. The standard drug susceptibility method is culture-based, either on solid or liquid media (1, 29), and completion takes weeks to months during which time the patient may be suboptimally treated. Genotypic assays that detect mutations that confer resistance to injectable and fluoroquinolone antibiotics are emerging, such as assays for gyrA for fluoroquinolones and rrs and eis for injectables. However sensitivity and specificity of these mutations appear to reside in the 80-90% range and interpretation of these mutations is complex and best made with precise sequencing results (8). For other drugs such as para-aminosalicylic acid, cycloserine, ethionamide, and linezolid genotypic testing does not exist in any current form. Therefore more rapid methods of phenotypic drug susceptibility testing are greatly needed.

For many years, bacteriophages have been as research tools to enhance understanding of microbial pathogens. Recently, they have been applied in bacterial detection assays to rapidly detect the presence of viable bacterial cells (19). For example, D29 mycobacteriophage has been shown to successfully infect and detect the presence of viable M. tuberculosis cells (19, 22). The bacteriophage is specific to mycobacterium through recognition of host surface proteins from which it infects. D29 mycobacteriophage is advantageous in detecting slow-growing mycobacteria because it invades the host cell in seconds and begins DNA replication within minutes (5, 27). Studies with M. smegmatis have shown release of D29 progeny phage as early as 90 minutes after infection (9, 22).

Several mycobacteriophage-based assays have been attempted for both detection and drug susceptibility testing of Tb. Two main approaches have been used: detection of progeny phages using sensor cells such as M. smegmatis (21), and detection of reporter constructs generated by engineered phages, such as luciferase-producing phages (3, 4, 14, 21). Certain phage-based assays have been commercialized, such as FASTPlaqueTB (17) which uses D29 phage for detection of M. tuberculosis directly from sputum and FastPlaque-Response, which detects rifampin resistance (15). Readout of these latter assays entails lawns of sensor cells. Luciferase reporter mycobacteriophages have been used to obtain DST (Drug Susceptibility Test) information for isoniazid, rifampin, streptomycin, and ethambutol with a median turnaround time of 3 days (2), however the assay requires manipulating liquid cultures of Tb in a luminometer which is not widely available, thus other engineered phage constructs are being tried (26). These phage-based approaches have reported only qualitative susceptible/resistant result.

Moreover, both molecular and conventional DST for TB yield a qualitative “susceptible” or “resistant” result for each drug, when in truth bacterial resistance to antibiotics is a quantitative spectrum.

There is a long felt need in the art for compositions and methods useful to identify and quantify susceptibility or resistance to a drug by means of a rapid, molecular, phenotypic susceptibility assay. More specifically, there is a long felt need for a quantitative metric of TB drug susceptibility-akin to the Minimum Inhibitory Concentration (MIC)—to help clinicians who often face difficult and limited choices. The present invention satisfies these needs.

### SUMMARY OF THE INVENTION

Applicants recently published a qPCR-based method (24) that utilized amplification of the M. tuberculosis 16S rRNA gene after 3 days of incubation with antituberculous drugs. The assay required use of propidium monoazide, a DNA-binding dye to decrease background amplification from killed organisms. It entailed several steps, including exposure to bright light and a wash that required strict biosafety. Other known methods for testing susceptibility of M. tuberculosis strains were also too slow. The present invention was made with the intent of finding quicker and better methods to test drug sensitivity of M. tuberculosis.

To that end, the present application discloses superior and faster phage-based methods for testing drug sensitivity of M. tuberculosis relative to what was known in the art. The present application describes herein development of a real-time PCR assay of phage DNA that can work on any of the 13 main antituberculous drugs without the need of sensor cells or engineered phages. The assay can also be used to test any other drugs that might be useful for treating tuberculosis. Real-time PCR is increasingly available to clinical laboratories worldwide. Therefore, it was also a goal of the present work to quantitate the real-time PCR cycle threshold as a maneuver to estimate the MIC of the organism.

The present invention provides compositions and methods for rapid phenotypic determination of drug susceptibility of M. tuberculosis, wherein one or more drugs can be tested in a short period of time.

The present application discloses a rapid phenotypic drug susceptibility assay that can be used with any drug and can be used to test a panel of drugs simultaneously. The assay of the present invention encompasses real-time PCR assays of phage DNA allowing quantitative results. Previous assays are slower, only provide qualitative results, often allow the testing of only one or two drugs, and some are only useful for detecting the presence of mycobacteria. One of ordinary skill in the art will appreciate that any method useful for detecting and quantifying the amount of phage present is encompassed by the present invention.

The present invention provides methods for determining which therapeutic agent or agents can be used to treat a subject with TB. A sample can be obtained from the subject, aliquots of the sample can be analyzed as described herein to determine susceptibility of the M. tuberculosis to a particular drug or drugs, and the results of the assay can be used by a clinician to determine which drugs to use to treat the subject, the dosages necessary, etc.

Based on the results of the diagnostic assays, in one embodiment, a subject may be treated with one drug alone. In another embodiment, other drugs may be used in a combination therapy with the first drug.

In one embodiment, the present invention provides compositions and methods for determining susceptibility of M. tuberculosis to one or more drugs. The method comprises testing a sample comprising M. tuberculosis by:

1) preparing at least one culture from the test sample;

2) contacting one culture with a drug and optionally contacting additional cultures with additional drugs, wherein each additional culture receives a different drug than the other cultures;

3) contacting a culture or additional cultures with a mycobacteriophage;

4) determining the amount of mycobacteriophage present after contacting the culture or additional cultures with mycobacteriophage;

5) comparing the amount of mycobacteriophage present in the treated cultures with the amount of mycobacteriophage present in an otherwise identical untreated culture;

6) wherein an increase in mycobacteriophage in an untreated culture relative to a culture treated with a drug is an indication that the M. tuberculosis present in the sample is susceptible to the treatment drug.

In one aspect, the culture is a suspension culture.

In one aspect, the mycobacteriophage is D29. In another aspect, it is L5.

The methods of the present invention are outlined, for example, in FIGS. 5, 6, and 7, and in Example 5, where protocols and standard operating procedures encompassed by the invention are provided.

In one aspect, the amount of D29 mycobacteriophage or other mycobacteriophage present is determined using real-time PCR. A decrease in real-time PCR cycle threshold (C_{T}) value is an indication of an increase in the amount of mycobacteriophage present.

In one aspect, the amount of mycobacteriophage present is determined using at least one reporter dye or at least one internal probe. In one aspect, the reporter dye is an intercalating dye such as SYBR green. In another aspect, an internal sequence-specific probe such as a TaqMan probe, Molecular Beacon probe, FRET probe, Scorpion probe, or other similar sequence-specific chemistry is used.

In one aspect, the cultures of M. tuberculosis are contacted with drug and D29 mycobacteriophage simultaneously. In another aspect, cultures are contacted with drugs prior to being contacted with D29 mycobacteriophage. In one aspect, the cultures are contacted with a drug for up to about 72 hours before being contacted with D29 mycobacteriophage. In another aspect, the cultures are contacted with a drug for up to about 48 hours before being contacted with D29 mycobacteriophage. In yet another aspect, the cultures are contacted with a drug for up to about 24 hours before being contacted with D29 mycobacteriophage.

In one embodiment, the cultures are in contact with D29 mycobacteriophage for up to about 72 hours. In one aspect, the cultures are in contact with D29 mycobacteriophage for up to about 48 hours. In another aspect, the cultures are in contact with D29 mycobacteriophage for up to about 24 hours before the measurement of D29 mycobacteriophage is begun.

Subjects with TB are typically treated with more than one drug. Although there are thirteen drugs commonly used for treating TB, the compositions and methods of the present invention encompass testing more drugs at a time than just those thirteen drugs. More than one drug can be tested at a time, including drugs that are not yet known to be useful for treating TB. Commonly used drugs for TB treatment which can be tested using the methods of the present invention include isoniazid (INH), streptomycin sulfate (SM), rifampin (RMP), pyrazinamide (PZA), ethambutol (EMB), ethionamide (ETA), capreomycin sulfate (CM), amikacin (AK), kanamycin sulfate (KM), levofloxacin, p-aminosalicylic acid (PAS), D-cycloserine (CS), clofazimine (CF), ofloxacin (OFX), moxifloxacin (MFX), and linezolid (LZD). In one aspect, any new investigational antitubercular drug can be tested or any other drug which may have antibacterial activity against M. tuberculosis. In one aspect, critical concentrations for use in the assay can be determined and the drug or additional drugs are added at their critical concentrations in the assay.

In one aspect, useful critical concentrations in μg/ml are:

| | |
|---|---|
| Isoniazid | 0.1 |
| Rifampin | 1.0 |
| Ethambutol | 5.0 |
| Streptomycin | 1.0-2.0 |
| Amikacin | 1.0 |
| Kanamycin | 1.0-5.0 |
| Capreomycin | 2.5 |
| Ofloxacin | 2.0 |
| Moxifloxacin | 0.25 |
| Ethionamide | 5.0 |
| para-aminosalicylic acid | 2.0 |
| Cycloserine | 30.0 |
| Linezolid | 1.0 |

The assays can be performed with multiple drugs at a time, although preferably only one drug per well or chamber being tested is used and at least one untreated control well or chamber is included in the assay. The assays can be easily set up using different kinds of devices or chambers for testing, including tubes, multiwell chamber plates, and other dishes. In one aspect, the methods of the invention are useful for high throughput assays. The practice of the invention is not limited to a specific number of wells per plate or to a specific number of tubes or other devices having chambers. A plate may comprise multiple wells or chambers for the process being performed. For example, the plate can be a 1 well, 6 well, 12 well, 24 well, 48 well, 96 well, 384 well, or 1536 well plate. For example, 96 well plates can be used (see FIGS. 5 and 6). The drugs can be tested using the same multiwell/multichamber device, using multiple tubes, or using multiple multiwell or multichamber devices. A multiwell chamber device includes, for example, a multiwell plate. The assays of the invention can be performed in any laboratory using culture-based DST that has access to a real-time PCR thermocycler.

One of ordinary skill in the art will appreciate that the amount of M. tuberculosis used and the amount of D29 mycobacteriophage or other mycobacteriophage used can be varied based on a variety of parameters and needs, including how quickly results are needed, how much M. tuberculosis is available in the sample, etc. For example, in one aspect, cultures of M. tuberculosis can be contacted with D29 mycobacteriophage at concentrations of about 10² pfu/ml to about 10⁴ pfu/ml. In one aspect, about 10³ pfu/ml are used. Of course, not just the concentration but the total amount of M. tuberculosis and D29 mycobacteriophage needed is also considered.

In one aspect, the PCR cycle thresholds are quantified. When quantified, in one aspect the PCR cycle thresholds are quantified amongst the test sample culture, the drug-treated sample culture, and the starting amount of bacteriophage. In one aspect, multiple drugs are tested and quantified.

In one aspect, the minimal inhibiting concentration of drug or additional drugs useful against the test mycobacterium is estimated.

In one aspect, analyses can be performed to determine ΔC_{T} and ΔC_{T} cut-off values. In one aspect, a Receiver-Operating Characteristic (ROC) analysis is performed using PASW Statistics Software or similar Software to define a cut-off in the ΔCt values that compares to agar proportion results. In one aspect, the cut-offs between the test sample culture that is not treated and the otherwise identical test sample cultures that are not treated with a drug are about +0.3 and −6.0, depending on the particular drug used. In one aspect, a significantly lower average ΔC_{T} is found among susceptible strains relative to resistant strains. In one aspect, a ΔC_{T} cutoff of +0.3 to −6.0 is obtained using ROC analysis.

In one aspect, the cut-offs yield at least about 80% accurate results. In another aspect, the cut-offs yield at least about 90% accurate results. In yet another aspect, the cut-offs yield at least about 95% accurate results. In a further aspect, the cut-offs yield at least about 98% accurate results. In one aspect, the cut-offs yield about 80% accurate results. In another aspect, the cut-offs yield about 90% accurate results. In yet another aspect, the cut-offs yield about 95% accurate results. In a further aspect, the cut-offs yield about 98% accurate results. In one aspect, ΔC_{T} cut-offs between the test sample cultures and the starting amount of mycobacteriophage are also evaluated as a quality control measure for the method of the invention.

One of skill in the art will appreciate that more than one drug can be tested simultaneously and the present application discloses testing multiple drugs at a time. The present application also discloses the use of multiwell plates to enhance speed and efficiency of drug susceptibility. For example, a single 96 well plate can be used to test all the known and approved TB drugs and controls. Moreover, additional wells and even plates can be used to vary parameters such as amount of bacteria or mycobacteria used, testing multiple concentrations of a drug, and for monitoring at different time points if sample size is small. Therefore, in one aspect, at least 2 different drugs can be tested at the same time. In another aspect, at least 4 different drugs are tested. In yet another aspect, at least 10 different drugs are tested. In a further aspect, at least 15 drugs are tested. In yet another aspect, at least 20 drugs are tested. In a further aspect, 13 different drugs are tested. In another aspect, 16 drugs are tested. In one aspect, when multiple drugs are tested the drugs are tested simultaneously in a multiwell device or in multiple chambers. In one aspect, the multiwell device is a multiwell plate. The number of wells or type of multiwell plate can be chosen to conform with the type of assay best suited for a laboratory based on the equipment it has, such as a reader or other instrument capable of using a 96 well plate. In one aspect, the type of plate used is selected from the group consisting of 6 well, 12 well, 24 well, 48 well, 96 well, 384 well, and 1536 well plates.

One of ordinary skill in the art will appreciate that the amount of mycobacteriophage present can determined at multiple intervals following contacting M. tuberculosis with mycobacteriophage. In one aspect, aliquots can be obtained from a culture at varied times and the amount of mycobacteriophage determined. In one aspect, the amount of mycobacteriophage is determined using real-time PCR.

The compositions and method of the invention are useful for testing different kinds of samples, including a sample from a subject suspected of having tuberculosis or a subject who has tuberculosis. Test samples from patients are typically sputum, but the present invention provides for the use of any sample that may comprise mycobacteria. In general, any biological sample such as sputum, CSF, serum, plasma, blood, other blood components, pleural effusion, gastric aspirates, urine, throat swabs, and stools can be used in the assay. In one aspect, a preferred sample is sputum. Assays can be performed starting with fresh samples from test subjects, although in some cases there may be a need to partially concentrate the specimen, purify the mycobacterium or to subject the mycobacterium present to a culture system to obtain enough mycobacteria for the assay. One of ordinary skill in the art will appreciate that the sample may have to be cultured to increase the number of mycobacteria before testing drug susceptibility.

Although the present invention provides an assay that is much faster than the other assays being used today, the assay can be set up for determinations at various times. In one aspect, susceptibility is determined in less than about 5 days. In another aspect, susceptibility is determined in less than about 4 days. In yet another aspect, susceptibility is determined in less than about 3 days. In a further aspect, susceptibility is determined in less than about 2 days, and in another aspect, in about 1 day or less.

One of skill in the art will appreciate that different primers can be used in the PCT assay of the invention. For example, PCR can be performed using primers selected from the group of primers having SEQ ID NOs:1-6.

SEQ ID NO: 1- AGCCGATCAGAAGCACGGGC
Forward Primer D29-F (5′-AGCCGATCAGAAGCACGGGC-3′)
SEQ ID NO: 2- AGCGGCTCTTAGGAGGGGCC
Reverse Primer D29-R (5′-AGCGGCTCTTAGGAGGGGCC-3′)
SEQ ID NO: 3- GCCACCAGGAGCCACGAAC
Forward primer: 5′- GCCACCAGGAGCCACGAAC-3′
SEQ ID NO: 4- AATAGGGAAGGAGTCTGCGTTTG
Reverse primer: 5′-AATAGGGAAGGAGTCTGCGTTTG-3′
SEQ ID NO: 5- CCACCAGGAGCCACGAACT
Forward primer 5′-CCACCAGGAGCCACGAACT-3′
SEQ ID NO: 6- AGTGGCGTAGATCACCTTGACA
Reverse primer: 5′-AGTGGCGTAGATCACCTTGACA-3′

The present invention further encompasses the use of an internal TaqMan Minor groove binding probe (SEQ ID NO:7) in conjunction with primers 5 and 6:

SEQ ID NO: 7- TATACCCCCGGAATCG
MGB Probe: FAM-5′-TATACCCCCGGAATCG-3′-MGB

In one embodiment, the sample comprising M. tuberculosis can be tested using the standard agar proportion method or other conventional drug susceptibility methods, for example, the BACTEC™ MGIT™ 960 Mycobacterial Detection System. The results of these methods can be compared with the results of the mycobacteriophage assay.

In one embodiment, the sample comprising M. tuberculosis can be tested using amplification of the M. tuberculosis 16S rRNA gene or other TB genes, including with propidium monoazide treatment, and the results of each method compared.

In one embodiment, a drug susceptibility profile is determined for the M. tuberculosis being tested based on the drugs being screened. In one aspect, the profile is used to select a drug treatment regimen for the subject from whom the M .tuberculosis was obtained.

In one embodiment, the present invention provides a method for treating tuberculosis in a subject, by first testing the subject suspected of having tuberculosis using the methods of the invention to determine the drug susceptibility of the strain of M. tuberculosis in the subject. Then the subject is treated with the drug or drug combination that the strain of M. tuberculosis is susceptible to. The assay of the invention will also reveal, for example, if the M. tuberculosis is multidrug resistant or extensively drug-resistant or “totally” drug resistant.

The present invention further provides kits for practice of the invention. In one aspect, the invention provides a kit for screening susceptibility of M. tuberculosis to one or more drugs, where the kit comprises, optionally, one or more of the following: one or more drugs, culture medium, PCR reagents and primers, at least one strain of M. tuberculosis for culturing, a mycobacteriophage, and an instructional material for the use thereof.

In one aspect, the assay is useful for identifying drug-resistant mycobacteria. In another aspect, the assay is useful for identifying susceptible mycobacteria. In one aspect, the assay is useful for identifying multidrug-resistant mycobacteria. In one aspect, the assay is useful for identifying extensively drug-resistant mycobacteria.

The amount of mycobacteria cultured and the amount of mycobacteriophage added can be varied in the assays as described herein.

The assays of the present invention are also useful for identifying new drugs to treat TB. By new drugs in this context is meant drugs that are not currently known to be useful for treating TB. Panels of test drugs which have not yet been used to treat M. tuberculosis can be screened against one or more strains of M. tuberculosis using the compositions and methods described herein for screening susceptibility of M. tuberculosis to drugs. Decreases in phage level in a treated group of a particular test drug relative to the level in the control untreated group is an indication that the test drug is useful for treating that strain of M. tuberculosis.

The amount of a given drug incorporated into a given volume of the medium of the present invention can be readily determined by those of skill in the art. This amount can be determined experimentally, for example by determining the critical concentration of a given drug by measuring the highest MICs of the tuberculosis drug for strains that are known to be susceptible to the drug, and the lowest MICs for clinical isolates that are known to be resistant to the drug. Ideally, the critical concentration of a drug is the concentration at which the majority of the drug-susceptible strains are inhibited, while the majority of the drug-resistant strains can grow. In one aspect, based on the critical concentration of drug in the medium, one can then determine the drug-resistance growth level or breakpoint, to be used when screening test sample cultures for drug susceptibility on the given drug (i.e., the percentage of growth on the drug as compared to growth in the absence of the drug; typically, when growth is above about 1% on the drug compared to growth in the absence of the drug, then the culture/M. tuberculosis is considered to be resistant to the drug).

Separate administration of each compound, at different times and by different routes, in some cases would be advantageous in treating a subject in need thereof. Thus, the components in the combination of the first line antitubercular drugs need not necessarily be administered at essentially the same time or in any order. The administration can be so timed that the peak pharmacokinetic effect of one compound coincides with the peak pharmacokinetic effect of the other.

In the setting of extensively drug-resistant (XDR) TB there are few “susceptible” agents and the choice of agent becomes one of “least-resistant” drugs. To that end, the present application discloses a rapid, quantitative, and phenotypic DST assay that could accommodate any TB drug. Disclosed herein is an assay where results are quickly obtainable and can be used in the triage process for rapid therapy of MDR patients while more traditional or conventional DST is pending. In one aspect, results can be obtained in less than 4 days. In one aspect, results can be obtained in less than 3 days. In another aspect, results can be obtained in less than 2 days. In yet another aspect, results can be obtained within one day. In one aspect, results can be obtained in 1-3 days.

In one embodiment of the invention, the present application provides compositions and methods useful for a rapid, molecular, phenotypic drug susceptibility assay for first and second line antituberculosis drugs.

In one embodiment, the assay is a D29 qPCR assay as disclosed herein. In one embodiment, the assay is a quantitative assay.

One of ordinary skill in the art will appreciate that the assays can be modified using mycobacteriophage other than D29, as long as the desired activity is present.

One of ordinary skill in the art will appreciate that other primers can be prepared and used to practice the methods of the invention because the phage sequence is known.

Various aspects and embodiments of the invention are described in further detail below. The application incorporates by reference other methods described in the art, such as those in Heifets et al. (U.S. Pat. No. 6,951,733) and Chou et al. (U.S. Pat. No. 7,601,831).

### BRIEF DESCRIPTION OF THE DRAWINGS

FIG. 1 (comprising FIGS. 1A-1E and FIG. 1D comprising 1D(1)-1D(6)): Development of the D29 phage-PCR assay for TB drug susceptibility testing. (A) 10³ pfu/ml phage alone, phage plus control Tb (10⁵ cfu/ml), and phage plus Tb pre-treated with drugs for 72 hours were compared. All samples were boiled and subjected to qPCR for phage DNA. Raw data are shown. (B) Durations of 6 and 24 hours of phage incubation with 72 h drug-treated H37Rv were compared. (C) Phage concentration was evaluated by comparing 10³, 10⁴, and 10⁵ pfu/ml of phage added to 72 h-treated H37Rv and MDR-TB, followed by 24 hours phage incubation. (D) Duration of drug treatment was evaluated by comparing 24 hours, 48 hours, and 72 hours of drug treatment of H37Rv and MDR Tb prior to 24 hours of 10³ phage incubation. Phage qPCR Ct, plaque counts, and Tb CFU were evaluated. Real-time PCR Ct was compared to D29 phage plaque counts and bacterial colony counts as a function of drug-treated time over 1-3 days followed by 24 hr phage incubation. (E) Durations of drug pre-treatment prior to phage versus drug co-treatment with phage were compared as indicated. Experiments in B, C, and E were performed in duplicate and data shown as mean ΔCt (Ct Tb/phage−phage alone). *, P<0.05; NS, P not statistically significant.

FIG. 2: Correlation between D29 phage-qPCR results and standard agar proportion method using the co-treatment method. Thirty-three M. tuberculosis isolates were tested by phage-qPCR and agar proportion assay for susceptibility to Rifampin, Streptomycin, Amikacin, Kanamycin, Capreomycin, Ofloxacin, Moxifloxacin, Linezolid, and Cycloserine. For the phage qPCR assay TB were co-incubated with drugs or control media and phage for 24 hours prior to boiling and qPCR. All phage assays performed in duplicate and mean qPCR Ct is shown. Cut-off threshold values were determined by ROC analysis. The red symbol x, − indicates a standard assay result that was discrepant with D29 phage-qPCR. Red symbols appear dark/black in the black and white version of the drawings.

FIG. 3: Correlation between D29 phage-qPCR results and standard agar proportion method using the pre-treatment method. Thirty three M. tuberculosis isolates were tested by phage-qPCR and agar proportion assay for susceptibility to Isoniazid, Ethambutol, Ethionamide, and para-aminosalicylic acid. For the phage qPCR assay Tb were pre-treated with drugs or control media for 48 hours, followed by phage incubation for 24 h, then boiling and qPCR. All phage assays performed in duplicate and mean qPCR Ct is shown. Cut-off threshold values were determined by ROC analysis. The red symbol x, − (black in the black and white graphs) indicates a standard assay result that was discrepant with D29 phage-qPCR.

FIGS. 4A-4H: Correlation between D29 phage-qPCR ΔCt and minimal inhibitory concentration. Thirty-three TB strains were tested by Sensititre®MYCOTB for MIC. The correlation of MIC values with ΔCt was shown for the indicated drugs. Red line indicates critical concentration. All phage-qPCR results were performed on duplicate cultures and mean±SD values are shown. Best-fit lines and Pearson regression R² values are shown. Amikacin, streptomycin, and para-aminosalicyclic acid exhibited a statistically significant correlation are not shown because the isolates did not exhibit a range of MICs.

FIG. 5: Flow Chart 1—comprises 5A to 5C: the chart demonstrates schematically drug panel array 1 (RIF, STR, AMK, KAN, CAP, OFX, LZD, CS) (5A), bacterial inoculum (5B), and D29 phage inoculum and collection of DNA (5C).

FIG. 6: Flow Chart 2—comprises 6A to 6C: the chart demonstrates schematically drug panel array 2 (INH, EMB, ETH, PAS) (6A), bacterial inoculum (6B), and D29 phage inoculum and collection of DNA (6C).

FIG. 7: Flow Chart illustrating the Protocol for Example 3 using sputum of TB patients.

FIG. 8: Graphic illustration of sediment experiments.

### DETAILED DESCRIPTION

### Abbreviations and Acronyms

cfu—colony forming unit

CSF—cerebrospinal fluid

ΔCt—correlation between Ct values (Ct control−Ct drug); (also referred to as ΔC_{T})

Ct—cycle time (threshold)

DST—drug susceptibility testing

H37Rv—mycobacteria strain

hr—hour (also referred to as “h”)

HA—Hemagglutination

HIA—hybridization induced aggregation

ICS—Infection-causing strains

KFOR—2 ketoglutarate oxidoreductase

M. tuberculosis—Mycobacterium tuberculosis

M7H9—Middlebrook 7H9

MGIT—mycobacteria growth indicator tube

MIC—minimal inhibiting concentration

MDR—multidrug-resistant

MDR TB—multidrug-resistant tuberculosis

OADC—oleic acid-albumin-dextrose-catalase

pfu—plaque forming unit

PMA—propidium monoazide

ROC—receiver-operating characteristic

TB—tuberculosis (also referred to as “Tb”; in reference to culturing, “Tb” refers to culturing m. tuberculosis)

TE—Tris-EDTA

TSB or TSA—Trypticase soy broth or agar

XDR—extensively drug resistant

### Drug Abbreviations

AK—amikacin (also referred to as AMK)

CF—clofazimine

CM—capreomycin sulfate (also referred to as CAP)

CS—D-cycloserine

EMB—ethambutol hydrochloride

ETA—ethionamide (also referred to as ETH)

KM—kanamycin sulfate (also referred to as KAN)

INH—isoniazid

LZD—linezolid

MFX—moxifloxacin

OFX—ofloxacin

PAS—para-aminosalicyclic acid

PZA—pyrazinamide

RMP—rifampin (also referred to as RIF)

SM—streptomycin sulfate (also referred to as STR)

### DEFINITIONS

In describing and claiming the invention, the following terminology will be used in accordance with the definitions set forth below. Unless defined otherwise, all technical and scientific terms used herein have the commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the subject.

As used herein, “alleviating a disease or disorder symptom,” means reducing the severity of the symptom or the frequency with which such a symptom is experienced by a subject, or both.

As used herein, an “analog”, or “analogue” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biological sample,” as used herein, refers to samples obtained from a subject, including, but not limited to, sputum, CSF, blood, serum, plasma, gastric aspirates, throat swabs, skin, hair, tissue, blood, plasma, serum, cells, sweat and urine.

“Blood components” refers to main/important components such as red cells, white cells, platelets, and plasma and to other components that can be derived such as serum. In the context of the present invention, generally a blood component would be one useful as a sample for use in determining drug susceptibility of M. tuberculosis present in the sample.

As used herein, the term “carrier molecule” refers to any molecule that is chemically conjugated to the antigen of interest that enables an immune response resulting in antibodies specific to the native antigen.

A “chamber”, as used herein, refers to something to which a solution can be added, such as a tube or well of a multiwell plate, etc.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above. When referring to a compound of the invention, and unless otherwise specified, the term “compound” is intended to encompass not only the specified molecular entity but also its pharmaceutically acceptable, pharmacologically active analogs, including, but not limited to, salts, polymorphs, esters, amides, prodrugs, adducts, conjugates, active metabolites, and the like, where such modifications to the molecular entity are appropriate.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

The term “delivery vehicle” refers to any kind of device or material which can be used to deliver compounds in vivo or can be added to a composition comprising compounds administered to a plant or animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

By the term “determining the amount of D29 mycobacteriophage present” is meant performing an assay which either directly or indirectly calculates the amount of D29 mycobacteriophage present and can include measuring DNA, such as by using PCR.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

The terms “formula” and “structure” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound of the invention to reduce or impede a described function, such as having inhibitory sodium channel activity. Preferably, inhibition is by at least 10%, more preferably by at least 25%, even more preferably by at least 50%, and most preferably, the function is inhibited by at least 75%. The terms “inhibit”, “reduce”, and “block” are used interchangeably herein.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “modulate”, as used herein, refers to changing the level of an activity, function, or process. The term “modulate” encompasses both inhibiting and stimulating an activity, function, or process.

The term “new investigational antitubercular drug” refers to a drug approved for testing as a treatment for TB or for one being tested for its effects on M. tuberculosis.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The term “regulate” refers to either stimulating or inhibiting a function or activity of interest.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide that has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

By the term “suspected of having tuberculosis” in the context of this application is meant someone who has been diagnosed with tuberculosis or who because of symptoms expressed appears to have tuberculosis.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

As used herein, the term “treating” can include prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Chemical Definitions

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl.

As used herein, the term “optionally substituted” typically refers to from zero to four substituents, wherein the substituents are each independently selected. Each of the independently selected substituents may be the same or different than other substituents. For example, the substituents of an R group of a formula may be optionally substituted (e.g., from 1 to 4 times) with independently selected H, halogen, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain and amino acid.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like. “Optionally substituted aryl” includes aryl compounds having from zero to four substituents, and “substituted aryl” includes aryl compounds having one or more substituents. The term (C₅-C₈ alkyl)aryl refers to any aryl group which is attached to the parent moiety via the alkyl group.

“Heterocycle” refers to any stable 4, 5, 6, 7, 8, 9, 10, 11, or 12 membered, (unless the number of members is otherwise recited), monocyclic, bicyclic, or tricyclic heterocyclic ring that is saturated or partially unsaturated, and which consists of carbon atoms and 1, 2, 3, or 4 heteroatoms independently selected from the group consisting of N, O, and S. If the heterocycle is defined by the number of carbons atoms, then from 1, 2, 3, or 4 of the listed carbon atoms are replaced by a heteroatom. If the heterocycle is bicyclic or tricyclic, then at least one of the two or three rings must contain a heteroatom, though both or all three may each contain one or more heteroatoms. The N group may be N, NH, or N-substituent, depending on the chosen ring and if substituents are recited. The nitrogen and sulfur heteroatoms optionally may be oxidized (e.g., S, S(O), S(O)₂, and N—O). The heterocycle may be attached to its pendant group at any heteroatom or carbon atom that results in a stable structure. The heterocycles described herein may be substituted on carbon or on a nitrogen atom if the resulting compound is stable.

“Heteroaryl” refers to any stable 5, 6, 7, 8, 9, 10, 11, or 12 membered, (unless the number of members is otherwise recited), monocyclic, bicyclic, or tricyclic heterocyclic ring that is aromatic, and which consists of carbon atoms and 1, 2, 3, or 4 heteroatoms independently selected from the group consisting of N, O, and S. If the heteroaryl is defined by the number of carbons atoms, then 1, 2, 3, or 4 of the listed carbon atoms are replaced by a heteroatom. If the heteroaryl group is bicyclic or tricyclic, then at least one of the two or three rings must contain a heteroatom, though both or all three may each contain one or more heteroatoms. If the heteroaryl group is bicyclic or tricyclic, then only one of the rings must be aromatic. The N group may be N, NH, or N-substituent, depending on the chosen ring and if substituents are recited. The nitrogen and sulfur heteroatoms may optionally be oxidized (e.g., S, S(O), S(O)₂, and N—O). The heteroaryl ring may be attached to its pendant group at any heteroatom or carbon atom that results in a stable structure. The heteroaryl rings described herein may be substituted on carbon or on a nitrogen atom if the resulting compound is stable.”

The term “heteroatom” means for example oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The compounds of the present invention contain one or more asymmetric centers in the molecule. In accordance with the present invention a structure that does not designate the stereochemistry is to be understood as embracing all the various optical isomers, as well as racemic mixtures thereof.

The compounds of the present invention may exist in tautomeric forms and the invention includes both mixtures and separate individual tautomers. For example the following structure:

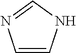

is understood to represent a mixture of the structures:

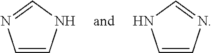 [SMILES: c1c[nH]cn1 ; c1c[nH]cn1]

The term “pharmaceutically-acceptable salt” refers to salts which retain the biological effectiveness and properties of the compounds of the present invention and which are not biologically or otherwise undesirable. In many cases, the compounds of the present invention are capable of forming acid and/or base salts by virtue of the presence of amino and/or carboxyl groups or groups similar thereto.

Compounds of the present invention that have one or more asymmetric carbon atoms may exist as the optically pure enantiomers, or optically pure diastereomers, as well as mixtures of enantiomers, mixtures of diastereomers, and racemic mixtures of such stereoisomers. The present invention includes within its scope all such isomers and mixtures thereof.

### EMBODIMENTS

In one embodiment, one or more drugs are used to test M. tuberculosis for susceptibility to the drug. Such drugs can include any drug that has been or will be identified as suitable for the inhibition of growth or the destruction of M. tuberculosis, and are therefore potentially useful for the treatment of tuberculosis. Such drugs can also be referred to herein as “tuberculosis drugs” or “antitubercular drugs”. Because some strains of M. tuberculosis are resistant to some tuberculosis drugs, in one embodiment it is desirable to test a sample containing M. tuberculosis from a subject with tuberculosis against a variety of drugs, including various doses of some drugs, in order to evaluate and select one or more drugs and doses that will be best for use in the test subject. If already identified, controls or standards can be used. Therefore, the present invention includes the incorporation of any tuberculosis drug into the medium of the present invention for the purpose of testing a sample of M. tuberculosis, or a test sample from a subject suspected of having M. tuberculosis, for susceptibility to the drug. Such drugs include, but are not limited to, isoniazid (INH), streptomycin sulfate (SM), rifampin (RMP), pyrazinamide (PZA), ethambutol (EMB), ethionamide (ETA), capreomycin sulfate (CM), amikacin (AK), kanamycin sulfate (KM), levofloxacin, p-aminosalicylic acid (PAS), D-cycloserine (CS), clofazimine (CF), ofloxacin (OFX), moxifloxacin (MFX), linezolid (LZD), and any new investigational antitubercular drug.

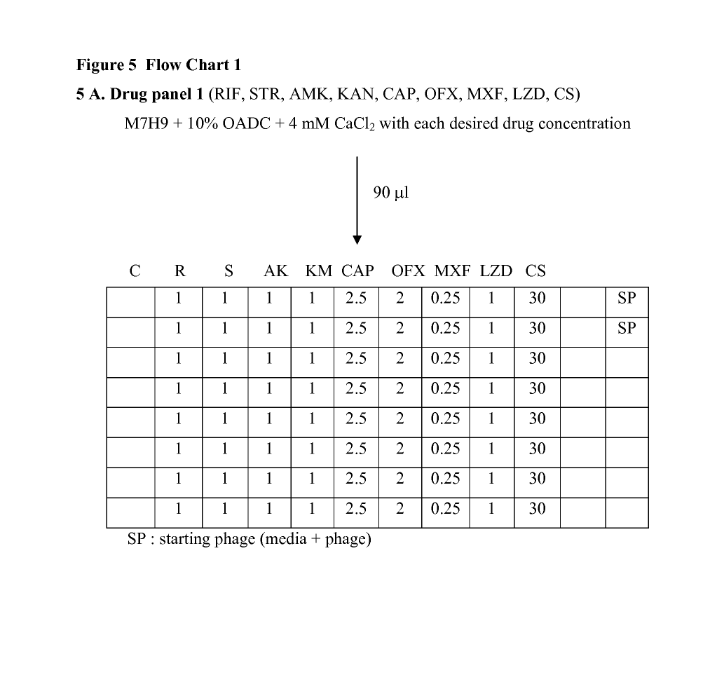

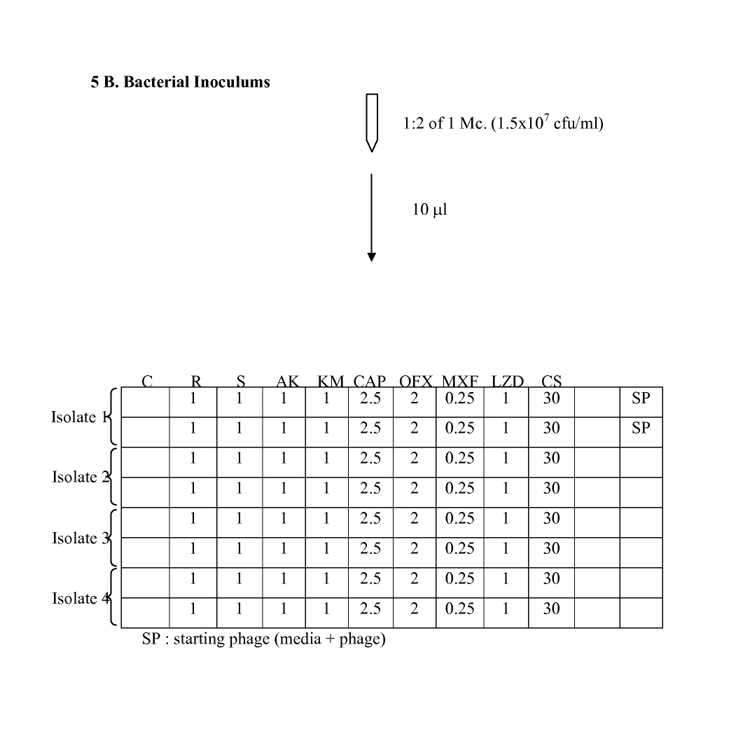

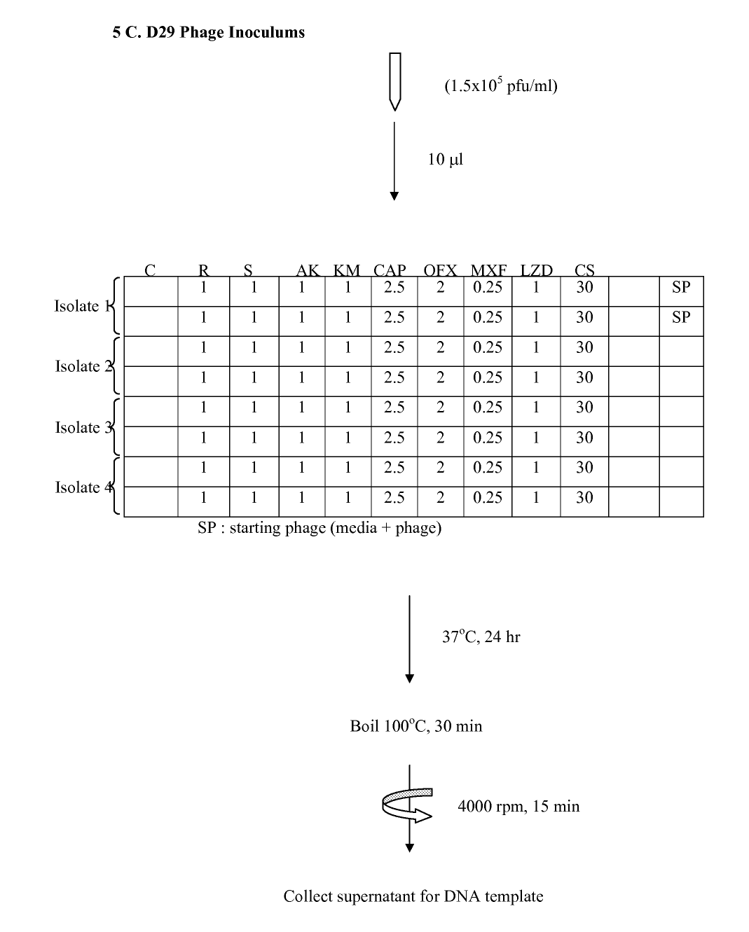

In one embodiment, the samples and cultures are prepared according to one or more of the protocols of FIGS. 5-7, including the inoculum, sedimentation, and treatment, as well as phage measurement assays.

Preferably, only one drug per test well or chamber is used. By using multiwell plates or multiple tubes, depending on the number of drugs being tested all tests can be performed at the same time and in the case of a multiwell plate such as a 96 well plate many drugs can be tested at the same time in the same plate. In one aspect, thirteen or more drugs can be tested at the same time. In one aspect, fifteen or more drugs are tested at the same time. In another aspect, twenty or more drugs are tested at the same time.

In one embodiment, the present application provides critical concentrations of drugs for susceptibility testing used for the D29 phage qPCR assay of the invention, and methods for determining the critical concentrations. The present invention therefore encompasses the use of multiple drugs and the critical concentrations for testing the drugs. The drugs can be tested as panels of drugs, for example, using multiwell plates.

In one embodiment, results of the real-time PCR assay of the invention can be compared with results of tests on the same samples and drugs using the standard agar proportion method or any other method used to measure drug susceptibility of mycobacteria.

The assay is useful for testing drugs and drug families including, but not limited to, quinolones, aminoglycosides, isoniazid, rifampin, ethambutol, streptomycin, amikacin, kanamycin, capreomycin, ofloxacin, moxifloxacin, ethionamide, para-aminosalicylic acid, linezolid, and cycloserine. One of ordinary skill in the art, based on the compositions and methods disclosed herein, will appreciate that the present assay is useful for testing not just drugs known to potentially effect M. tuberculosis, but can also be used to assay for similar activity in other drugs as well.

The invention further encompasses the use of kits. Kits may comprise various containers for the formulations, including, but not limited to, vials, tubes, and multiwell plates. Kits may comprise multiple samples of each formulation. Multiple drugs or panels of drugs can be included in a kit. In an assay system of the invention, the kit further comprises buffers. These buffers are easily purchased from commercial suppliers. In general, the assay system of the invention can reduce the whole process of determining drug susceptibility of the sample M. tuberculosis being tested.

### EXAMPLES

### Example 1

### Materials and Methods

Mycobacterial Strains and Culture Conditions.

Mycobacterial strains used in this study included M. smegmatis (ATCC 607), M. tuberculosis H37Rv (ATCC 27294) and 32 clinical isolates confirmed as M. tuberculosis complex by a sequence specific FRET probe (11). These included 9 susceptible strains, 22 MDR Tb, and 1 XDR Tb obtained from the Mycobacteriology Service Unit, Department of Microbiology, Faculty of Medicine Siriraj Hospital, Mahidol University, Bangkok, Thailand. All work was approved by the University of Virginia Institutional Biosafety Committee and Human Investigation Committees. Tb isolates were cultured on Lowenstein-Jensen medium at 35° C. for three weeks. Cell suspensions were prepared in Middlebrook 7H9 (M7H9) broth supplemented with Middlebrook OADC enrichment (Difco, Livonia, Mich., USA) and adjusted to 0.5 McFarland for Trek Sensititre MYCOTB assay and 1.0 McFarland for the agar proportion method. For the quantitative PCR experiments, cell suspensions were prepared in M7H9 broth with OADC and incubated overnight at 35° C. and adjusted to 0.5 McFarland.

Antimicrobial Agents.

Drugs used were isoniazid (INH), rifampin (RIF), streptomycin sulfate (STR), kanamycin sulfate (KAN), ofloxacin (OFX), ethionamide (ETH), para-aminosalicylic acid (PAS), and D-cycloserine (CS; all from Sigma-Aldrich, St. Louis, Mo., USA); ethambutol hydrochloride (EMB), amikacin (AMK), capreomycin sulfate (CAP; all from MP Biomedicals, Solon, Ohio, USA); moxifloxacin (MXF; 400 mg/250 ml in NaCl injection form), and linezolid (LZD; 600 mg/300 ml in water injection form, both from University of Virginia Pharmacy, Charlottesville, Va., USA). INH, EMB, AMK, KAN, STR, CAP, and CS were dissolved in sterile distilled water. RIF and ETH were dissolved in dimethyl sulfoxide. OFX was dissolved in 0.1 N NaOH and PAS was dissolved in ethanol. All stock solutions were stored in single use aliquots at −80° C.

Standard Agar Proportion Method.

Antimicrobial susceptibility tests were carried out in Middlebrook 7H10 agar (Difco) according to standard procedure (20). Briefly, a 1.0 McFarland suspension was diluted 10-fold serially in sterile distilled water and dilutions of 10⁻² and 10⁻⁴ were inoculated onto M7H10 with and without drug and incubated at 35° C. Where available, critical concentrations endorsed by WHO (29) were used. Results were read 21 days after inoculation of media. At the critical concentration of each drug bacterial growth was counted and calculated for the % resistant colonies, whereby ≧1% is defined as resistance. H37Rv, susceptible to all drugs tested, was used for quality control.

Sequencing of Loci.

DNA was isolated from 2 weeks of Tb culture. Briefly, 1 ml of 1.0 McFarland suspension was transferred to 2 ml screw cap tube, centrifuged at 21,000×g for 15 min, and the pellet re-suspended in 500 μl Tris-EDTA (TE) buffer, boiled at 100° C. for 30 min, centrifuged at 21,000×g for 5 min, and supernatants stored at −20° C. for template. The following seven loci were amplified by PCR: rpoB (RIF), katG and inhA (INH), embB (EMB), gyrA (OFX, MXF), rrs (KAN, CAP, and AMK), and eis (KAN) using the locus-specific primers of Campbell et al (8). Each 25-μl PCR mixture contained 12.5 μl HotStarTaq master mix (Qiagen), 0.15 μl of the forward and reverse 50 μM primers, 7.2 μl nuclease free water, and 5 μl of genomic DNA. PCR was performed on an MyCycler (Bio-Rad, Hercules, Calif., USA) included an initial denaturation step at 95° C. for 15 min, followed by 35 cycles of denaturation at 95° C. for 30 sec, annealing at 60° C. for 30 sec, and elongation at 72° C. for 30 sec, with a final elongation step at 72° C. for 7 min PCR products were analyzed on 2% agarose-gels, verified PCR products were purified using MinElute® PCR Purification Kit (Qiagen) followed the manufacturer's protocol. Purified PCR products were measured spectrophotometrically, diluted with nuclease free water and mixed with primers then submitted to GeneWiz (GeneWiz.com) for DNA sequencing.

Trek Sensititre MYCOTB Assays.

The minimal inhibitory concentration (MIC) were determined using the Sensititre® MYCOTB kit (TREK Diagnostic systems, West Sussex, UK) according to manufacturer's procedure. Briefly, 100 μl of 0.5 McFarland Tb suspensions was transferred into a tube of 10 ml 7H9 broth supplemented with OADC to yield inoculums of 1×10⁵ CFU/ml, 100 μl was transferred into each well of a 96 well plate coated with and without several concentration of each drug, all wells were covered with an adhesive seal and incubated aerobically at 35° C. for 10 days. Per the manufacturer's instructions, growth was evaluated visually at 10 days and if positive control growth was not apparent then the incubation was extended to 21 days. The MIC was recorded as the lowest antibiotic concentration that reduced visible growth.

Propagation and Quantification of D29 Phage.

D29 phage (Biotec Laboratories Ltd., Ipswich, UK) was propagated following the standard protocol (18). Briefly, 10% volume of stationary phase M. smegmatis was added to M7H10+10% OADC and poured into Petri dish, and 100 μl of approximately 4×10³ pfu/ml D29 phage was spread. Plates were incubated at 35° C. overnight and 10 ml M7H9 with 10% OADC and 4 mM CaCl₂ was added and re incubated. Broth was removed and filtered through sterile 0.45 μm filters, aliquoted, and stored at 4° C. For quantification, serial dilutions were made and 10 μl of phage was mixed with 200 μl M. smegmatis and melted M7H10 agar, poured onto M7H10 plates, and incubated at 35° C. for 24-48 h. Visible plaques were counted.

Optimization of D29 Phage Assay Conditions.

H37Rv was used to optimize conditions. For antimicrobial killing, Tb were diluted in M7H9 plus 10% OADC and 4 mM CaCl₂ with drug at critical concentrations and incubated at 35° C. for three days. D29 phage was added into live and drug treated Tb followed by phage incubation at 35° C., DNA extraction, and qPCR. The optimal phage concentration and phage incubation duration was varied as indicated. For Tb cfu counts, serial dilutions were made and 10 μl was dropped on M7H10 agar and incubated at 35° C. for 3 weeks. Plaque counts were performed as above.

Evaluation of Optimized D29 Phage Assay.

33 isolates of M. tuberculosis were tested for drug susceptibility using D29 phage assay. Thirteen drugs including INH, RIF, EMB, STR, AMK, KAN, CAP, OFX, MXF, LZD, ETH, PAS and CS were prepared using recommended critical concentrations for MGIT960 broth media (29) with 10% OADC and 4 mM CaCl₂. For RIF, STR, AMK, KAN, CAP, OFX, MXF, LZD, and CS, 90 μl of each drug was transferred to a 96 well PCR plate, 0.5 McFarland Tb suspensions were diluted 1:10 in M7H9 with 10% OADC and 4 mM CaCl₂ to yield inocula of 1×10⁶ cfu/ml. Ten μl of Tb suspensions were added into each well followed by 10 μl of D29 phage (1×10⁴ pfu/ml) and incubated at 35° C. for 24 hr. For INH, EMB, ETH, and PAS, Tb cells were incubated with drug at 35° C. for 48 hr prior to adding D29 phage followed by incubation at 35° C. for 24 hr. However, much shorter times than 48 hours can be used for these drugs as well (data not shown).

D29 Phage qPCR Assay.

D29 phage infected cells were incubated in a heating block at 100° C. for 30 min then centrifuged at 8000×g for 5 min, with supernatant used as DNA template. The primer D29-F (5′-AGCCGATCAGAAGCACGGGC-3′) (SEQ ID NO: 1) and D29-R (5′-AGCGGCTCTTAGGAGGGGCC-3′) (SEQ ID NO: 2) were designed to amplify a 225-bp untranslated region within the D29 phage genome. These primers did not amplify M Tb DNA alone. PCR mixtures (25 μl) consisted of 12.5 μl of 2×iQSYBR Green Supermix (Bio-Rad, Hercules, Calif., USA), 0.25 μl of 50 mM forward primer and 0.25 μl of 50 mM reverse primer, 7 μl nuclease-free water and 5 μl DNA template. Each set of samples included a nuclease-free water negative control. PCR was performed on an iCycler (Bio-Rad, Hercules, Calif., USA) with initial denaturation at 95° C. for 10 min, followed by 40-cycle denaturation at 94° C. for 30 sec, annealing at 70° C. for 30 sec, and extension at 72° C. for 30 sec. Melting curve analysis was performed to confirm single amplicons.

Statistical Analysis.

Means were compared using t test or Mann-Whitney test. All Tb cultures were performed in duplicate. Data shown as mean or mean±SD. The correlation between Ct values (ΔCt) and MICs was calculated by Pearson correlation. Receiver-Operating Characteristic (ROC) analysis was performed with PASW Statistics Software and was used to define a cut-off in the ΔCt values that compared to agar proportion results. All P values were two-tailed.

### Results

Development of D29 Phage-qPCR Assay.

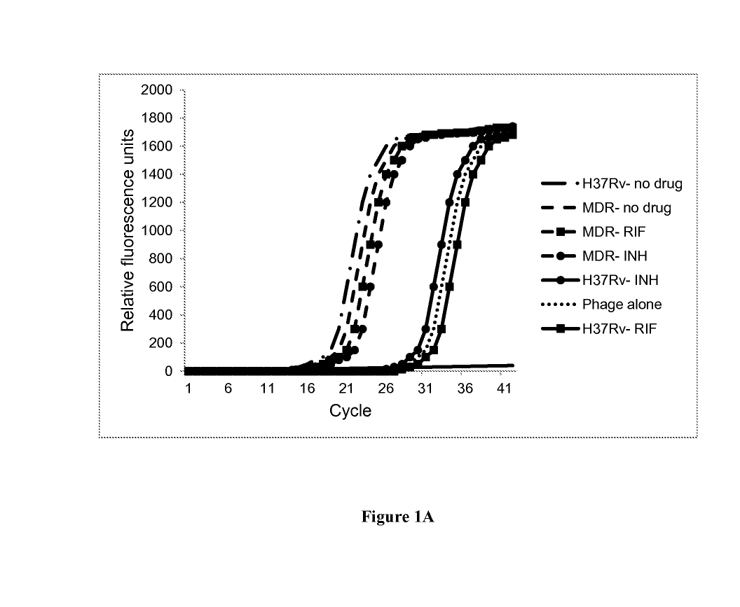

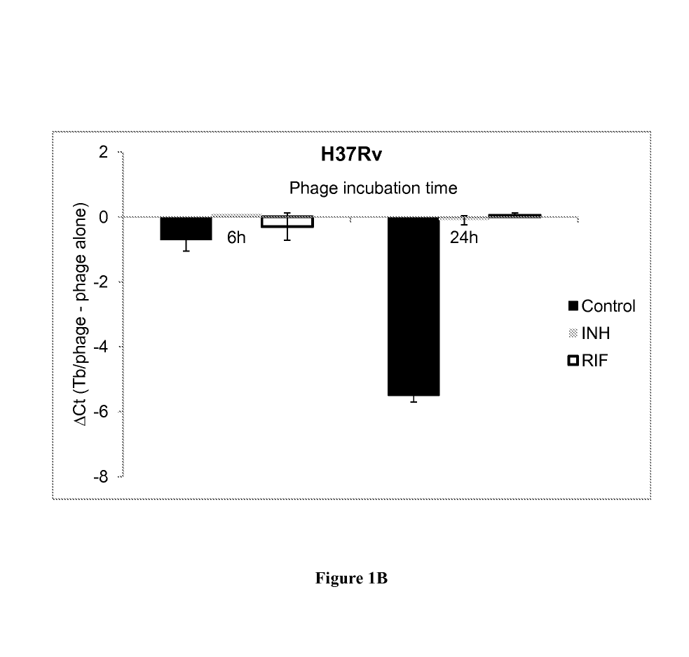

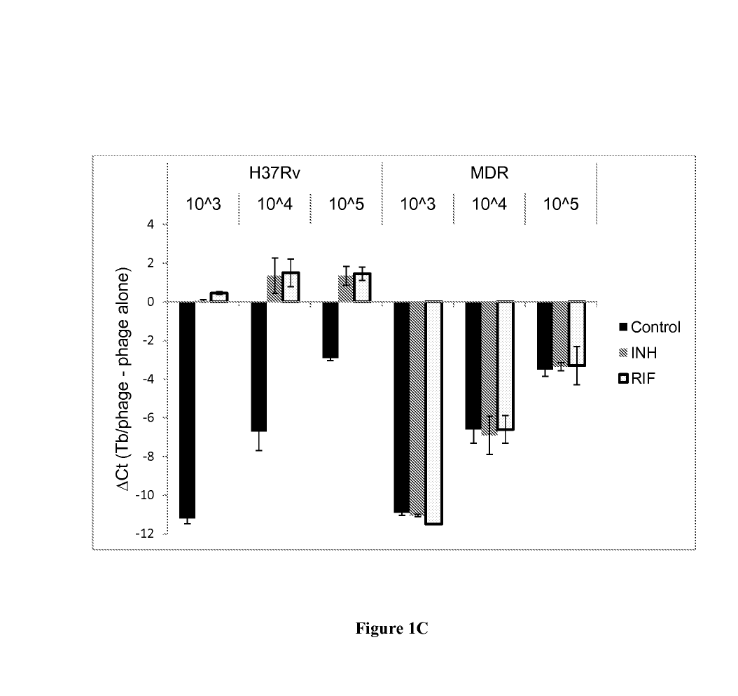

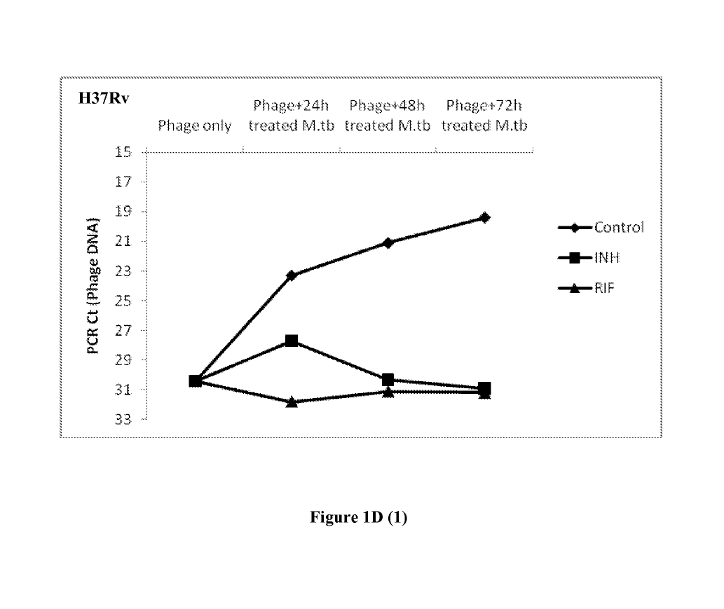

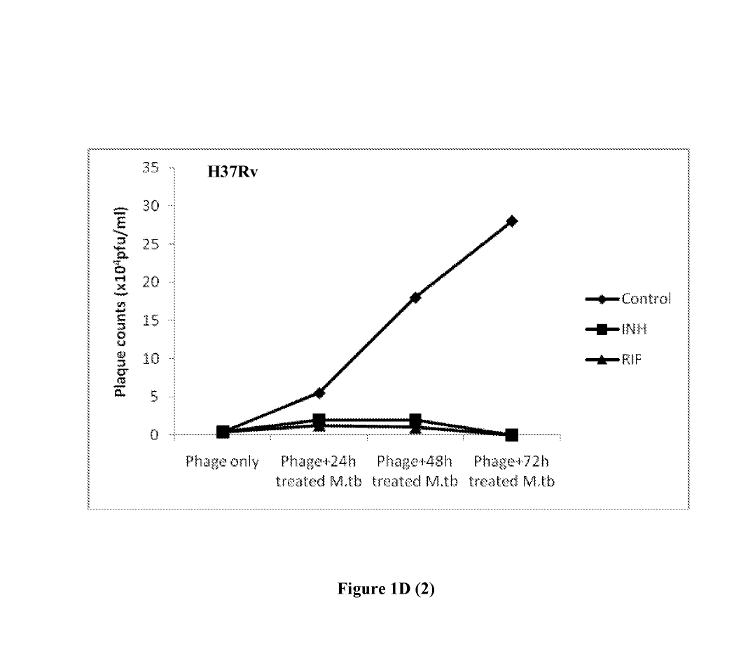

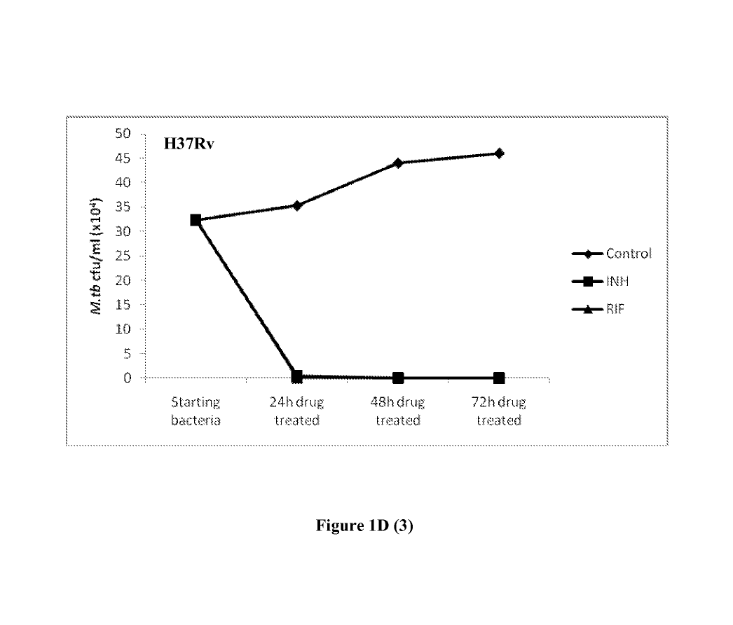

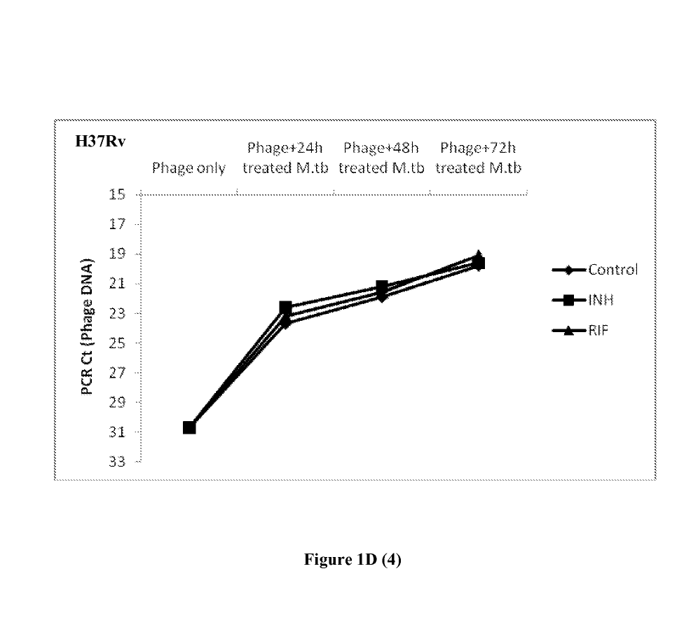

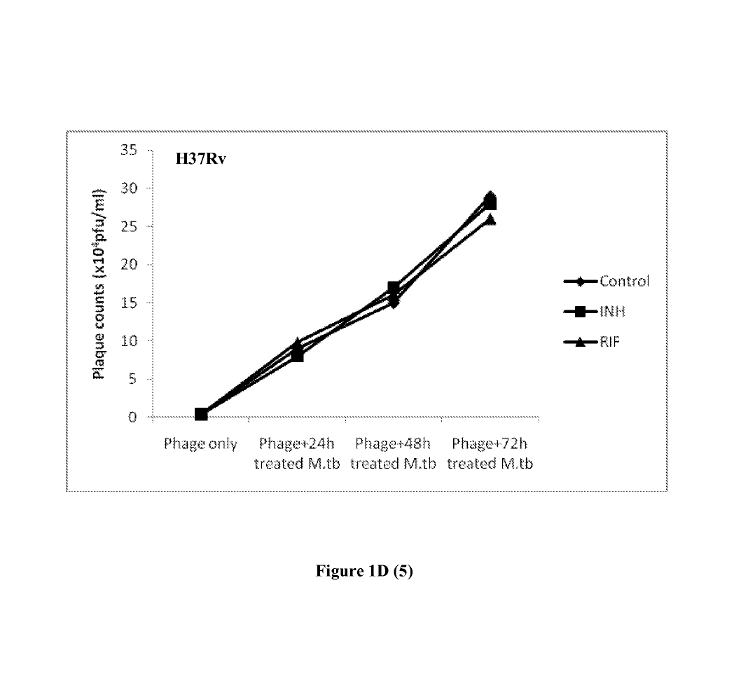

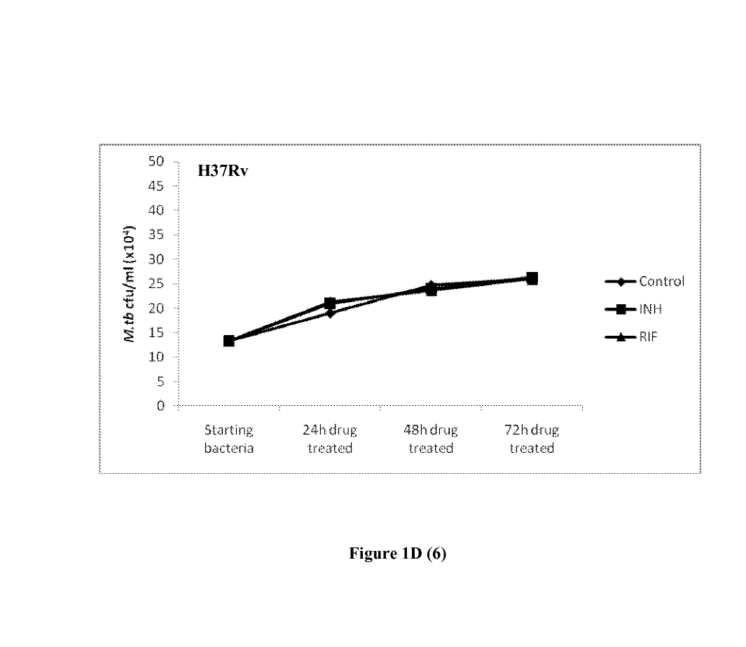

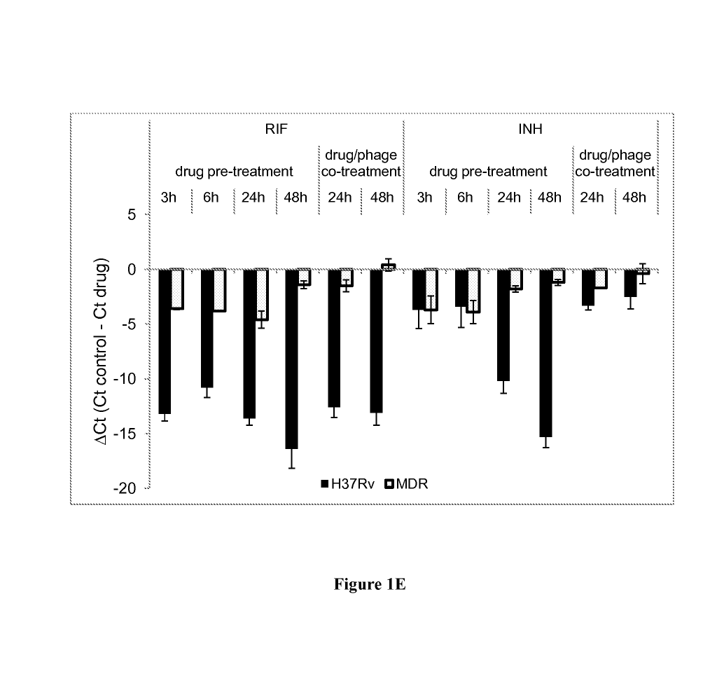

The hypothesis of our assay was that phage would infect and replicate their double stranded DNA in viable Tb cells, and that this DNA could be quantified by real-time PCR to discern Tb viability in the setting of drug treatment. To first examine this we performed qPCR for phage alone versus phage added to Tb grown in control media (FIG. 1A). One thousand pfu/ml of D29 mycobacteriophage and 10⁵ cfu/ml of Tb that were previously grown in control media for 72 h were used. Both H37 Rv and a clinical MDR strain (INH, RIF, EMB, STR resistant) were assayed and as expected in both instances there was a marked decrease in the real-time PCR cycle threshold (Ct) values reflective of increased quantities of phage DNA. Furthermore, upon INH or RIF pre-treatment of H37Rv, the number of phage did not increase/Ct value did not decrease. In contrast, the phage number increased/Ct value decreased when a MDR-Tb strain was pre-treated with INH or RIF. We then sought to optimize the parameters of the assay. Durations of phage incubation were compared and this revealed that 24 h of phage incubation was superior to 6 h of phage incubation as measured by a larger ΔCt (Tb/phage−phage alone) when using H37Rv grown in control media for 72 h (FIG. 1B). We then sought to examine the optimal phage inoculum for the assay and observed 10³ offered the largest ΔCt (Tb/phage−phage alone) versus 10⁴ or 10⁵ phage (FIG. 1C). The diminished ΔCt when 10⁴ or 10⁵ phage was used was due to elevated background/reduced Ct in the phage alone wells without commensurate reduction in Ct in the Tb/phage wells (data not shown). We then compared durations of Tb incubation. Both for H37Rv and MDR Tb, we observed that the phage Ct values decreased progressively with 24, 48, and 72 h of growth (FIG. 1D). As expected this corresponded to increased numbers of M. tuberculosis cfu/ml. Likewise the phage Ct values correlated with numbers of phage as quantified by conventional plaque counts on M. smegmatis indicator plates. For H37Rv, the qPCR Ct became clearly disparate with 24 h of rifampin treatment. In contrast, 48 h of INH treatment was required for a similar ΔCt (Ct control−Ct INH). We then more closely examined the durations required for pre-treatment of Tb with drugs prior to phage incubation, and the capacity for co-treatment of Tb with drugs and phage from the outset (FIG. 1E). This confirmed that INH pre-treatment was required and that 48 h was superior to 24 h, 6 h, and 3 h as measured by ΔCt (Ct control−Ct INH). In contrast, while 48 h of RIF pre-treatment was clearly able to discern drug susceptibility, so was 24 h, 6 h, 3 h, and 0 h of pre-treatment prior to 24 h phage incubation. In other words RIF, Tb inoculum, and phage could be added at once and yield a clearly disparate ΔCt in 24 h (ΔCt_{RIF} for H37Rv=−12 versus ΔCt_{RIF} for MDR=−1).

Evaluation of D29 Phage-qPCR Assay on Clinical Isolates.

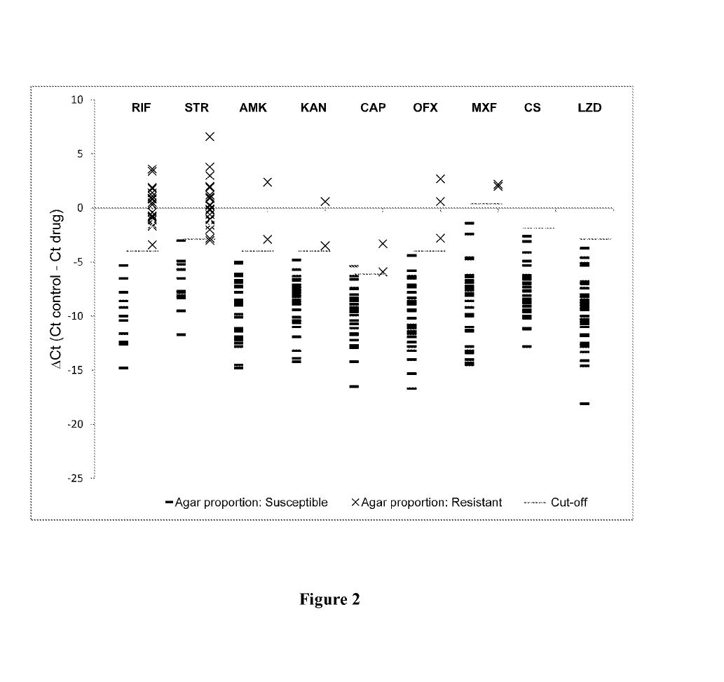

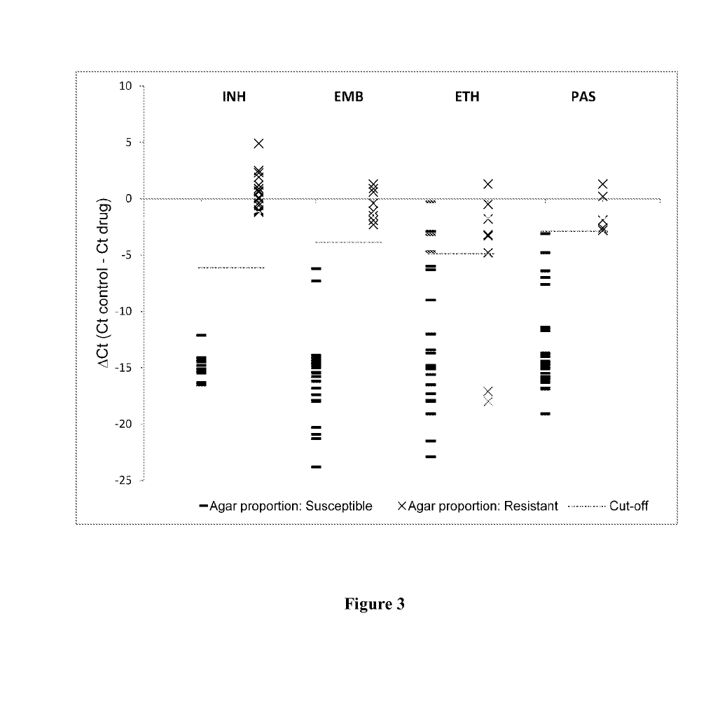

After the assay was developed with H37Rv and an MDR strain using RIF and INH we then examined its performance on all major anti-tuberculous medications in current use. We observed that the performance with EMB, ETH, and PAS behaved similar to INH, in that 48 hours of drug treatment of Tb prior to 24 hours of phage incubation yielded an optimal ΔCt in strains confirmed as drug susceptible by agar proportion method. However, other times can be used as well. In contrast, STR, AMK, KAN, CAP, OFX, MXF, LZD, and CS behaved similar to that of RIF in that no pre-treatment was required. Therefore drug, Tb inoculum, and phage could be added simultaneously and yield a disparate statistically significant ΔCt in 24 hour. For these drugs 48 hour of drug pre-treatment, akin to the INH method, also yielded a clearly disparate ΔCt, but we pursued the rapid 24 hour method in this work. In sum we applied the following conditions for 33 Tb strains: (1) 48 hr drug treatment followed by 24 hour of 10³ pfu/ml phage incubation for INH, EMB, ETH, and PAS and (2) simultaneous drug and 10³ pfu/ml phage incubation with Tb for 24 hours for RIF, STR, AMK, KAN, CAP, OFX, MXF, LZD, and CS. For these 33 strains we also scaled down the volume of the phage assay from 500 μl in tubes to 100 μl in a 96-well PCR plate. All 33 strains were tested using standard agar proportion method with the critical concentration for each drug as shown in Table 1, where resistance is defined as ≧1% growth of colonies on drug containing media (29). All strains were also characterized genotypically at the rpoB, inhA, katG, embB, gyrA, rrs, and eis loci (Table 51). The ΔCt (Ct control−Ct drug) was determined and compared to the conventional agar proportion results (FIGS. 2 and 3). We noted a significantly lower average ΔCt among susceptible strain versus resistant strains for all 13 drugs (P<0.05). We assigned a ΔCt cutoff of +0.3 to −6.0 for each drug using ROC analysis (Table 2) and these yielded accuracies of 100% for all drugs except capreomycin (97%) and ethionamide (82%). This reflected 1 false resistant capreomycin result very near the cutoff, 4 false resistant ethionamide results, and 2 false susceptible ethionamide results. We had few resistant isolates for linezolid, cycloserine, amikacin, capreomycin, ofloxacin and moxifloxacin, thus the cutoffs are not certain for these drugs.

Relationship of D29 Phage-qPCR and MIC Values.

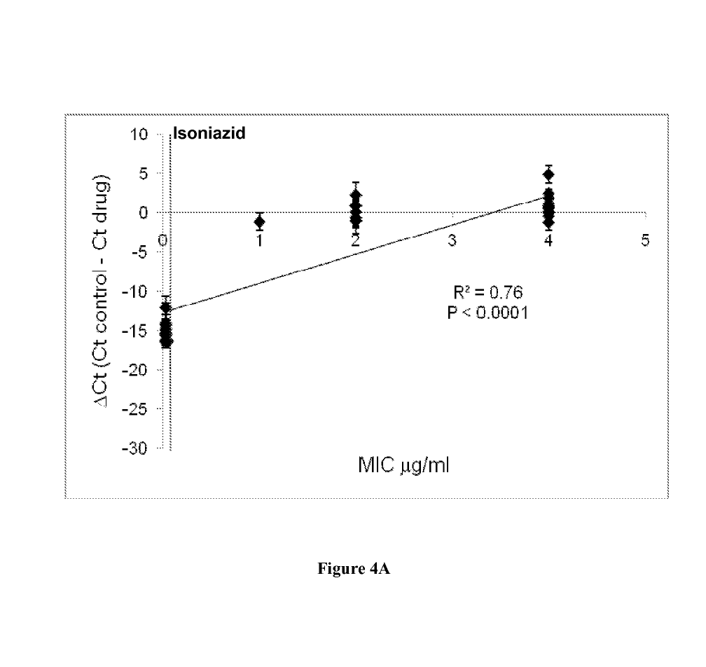

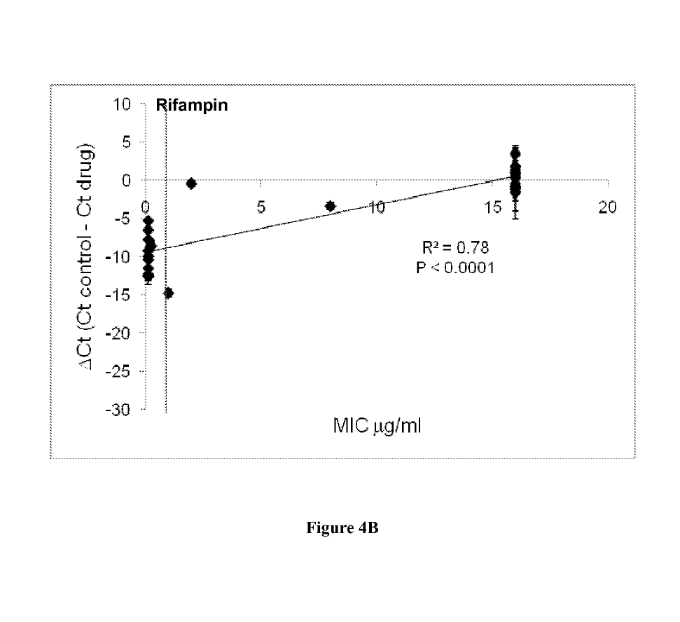

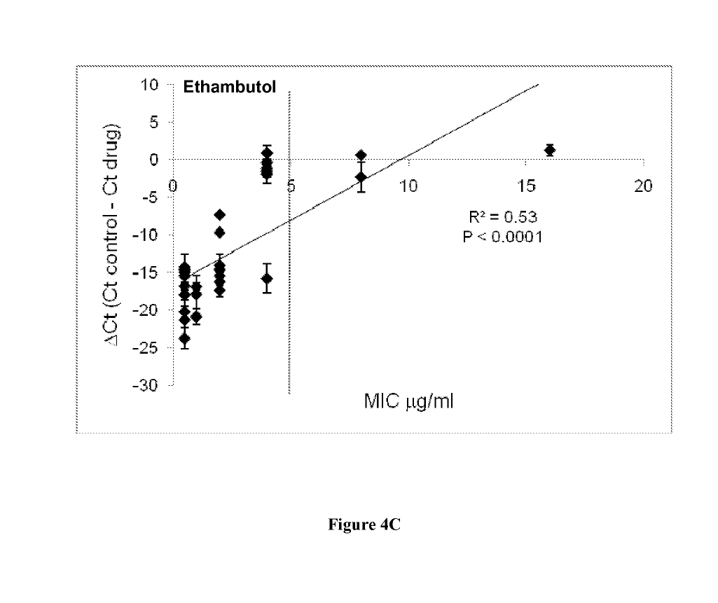

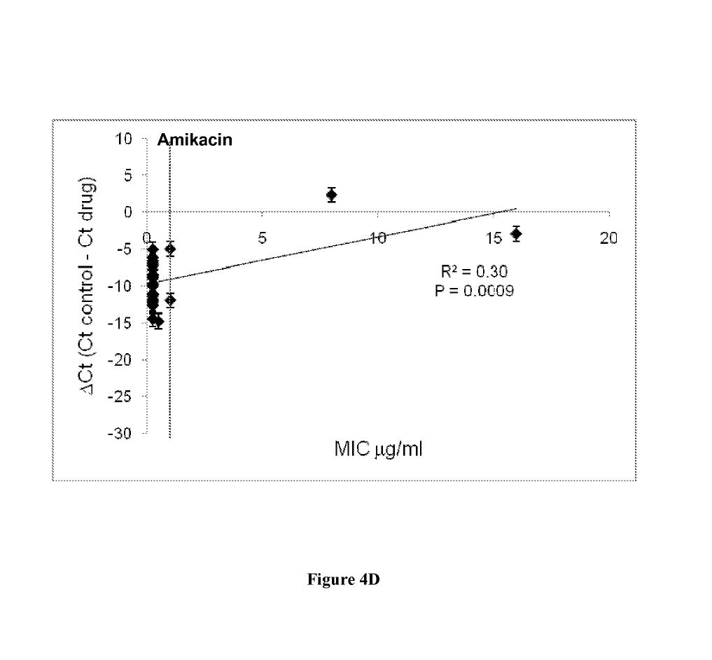

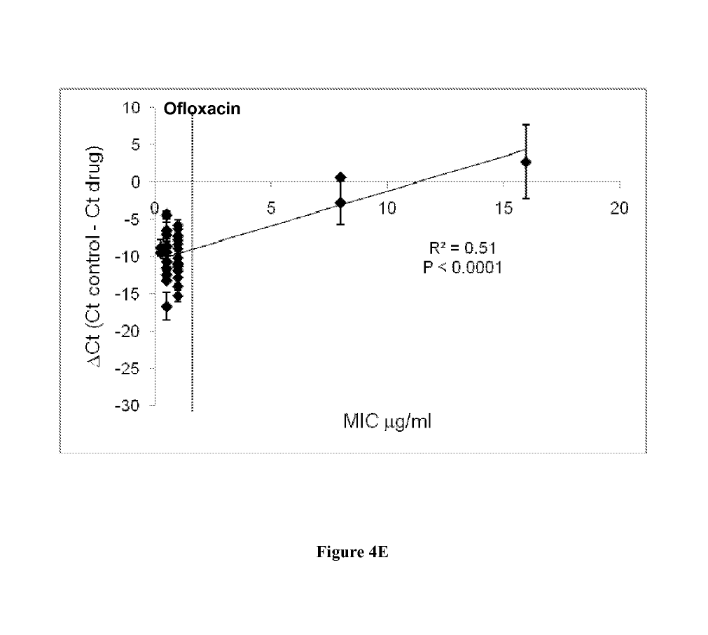

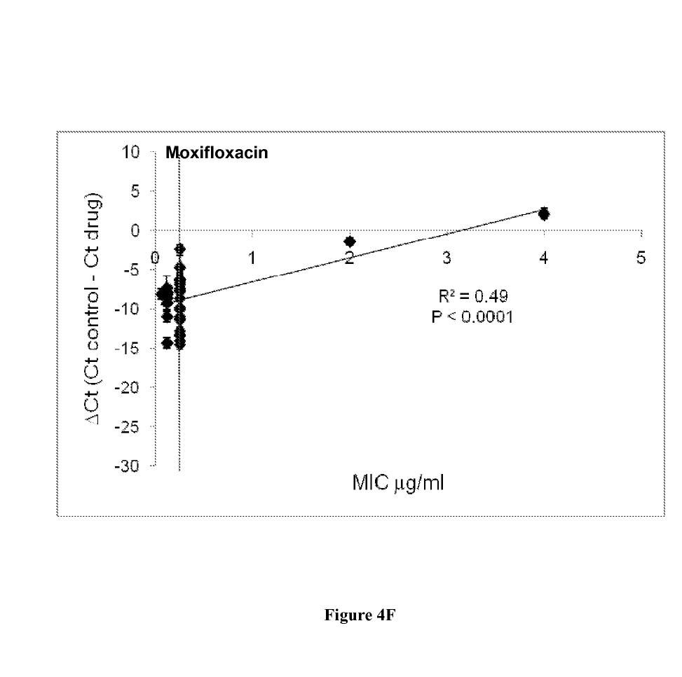

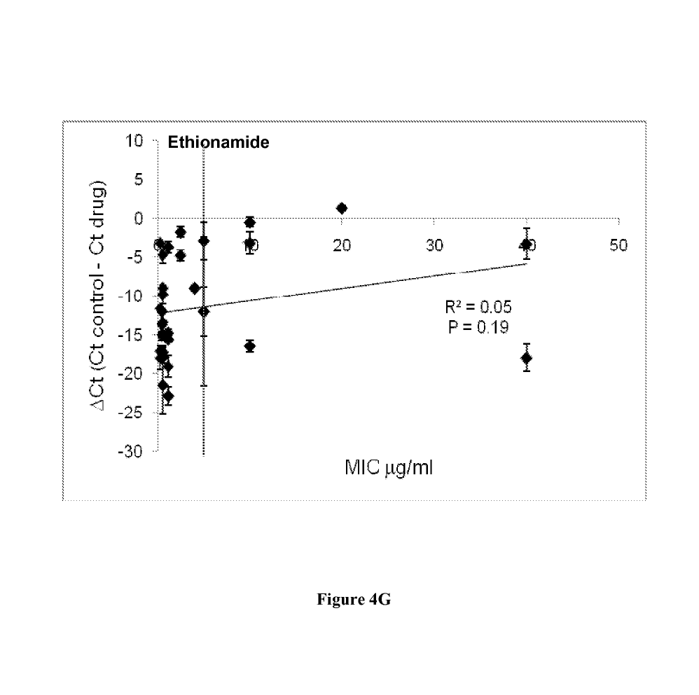

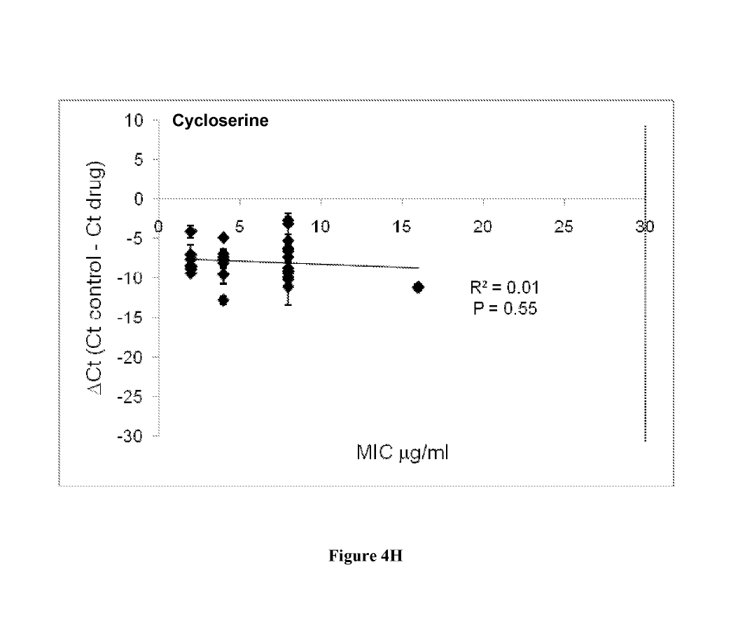

MICs for Tb isolates are very rarely pursued because they are time and resource intensive. We hypothesized that the quantitative ΔCt value might correlate with the MIC of the isolates: specifically, the wider the ΔCt between control and drug-treated cells the more susceptible the isolate should be to the drug. Thus for the 33 isolates we obtained MIC results by using the TREK Sensititre®MYCOTB plates, which offers MICs for INH, RIF, EMB, STR, AMK, KAN, OFX, MXF, ETH, PAS, and CS in a microplate format across a range of drug concentrations in M7H9 broth. Of note the MIC plate revealed 97% (383/396) concordant results vs. agar proportion method (1 false susceptible STR, 5 false susceptible EMB, 3 false resistant KAN, 3 false susceptible and 1 false resistant ETH). The correlation between phage-qPCR ΔCt (Ct control−Ct drug) and the MIC (FIG. 4) was statistically significant for INH, RIF, EMB, AMK, OFX, and MXF (P<0.001), with highest correlation for INH and RIF (R²=0.76−0.78). The phage-qPCR ΔCt did not statistically correlate with the MIC results for ETH or CS.

### Discussion

The present application discloses the development of a rapid 1 to 3 day phenotypic drug susceptibility test for Tb. The test provides results that are accurate compared to the agar proportion method for the 13 main antituberculous drugs and can permit one to construct an appropriate MDR or XDR Tb regimen rapidly. The resulting assay is faster, easier to use, more biosafe, and equally or more accurate than our recent PMA-qPCR approach (24). Indeed, for RIF, STR, AMK, KAN, CAP, OFX, MXF, LZD, and CS, we were able to obtain results in 1 day by co-treating Tb with drug and phage for 24 hours, followed by boiling and PCR. For the laboratory this means that if the wells are set up in a 96 well PCR plate it can be sealed, placed in the incubator, and then boiled in a heatblock, rendering it biosafe.

In contrast, as is the typical protocol for most phage-based DST methods, INH, EMB, ETH, and PAS required 48 hour of pre-treatment with drug prior to phage incubation for optimal use, but can be pre-treated for a shorter time. Presumably the differences among drugs in their ability to affect phage replication relate to their mechanisms of action. Since phage replication requires intact DNA, RNA, and protein synthesis machinery within the host cell, it is not surprising that fluoroquinolones (inhibit DNA synthesis), RIF (inhibits RNA polymerase (7)), and aminoglycoside injectables and LZD (inhibit protein synthesis) are able to quickly prevent phage DNA replication in our system. In contrast, INH, EMB (10), ETH, and to some extent PAS (which is also a folate antagonist (25)) are cell wall synthesis inhibitors, and we suspect inhibition of these mechanisms takes time for cell injury during which phage replication can occur, hence the requirement for 48 hour of pre-treatment. We are less able to explain the rapid activity of CS, whose mechanism of action is not clear but also appears to involve cell wall synthesis (6). We would emphasize that if one wishes to test all drugs with the 48 h pre-treatment approach, the ΔCt separation for RIF, STR, AMK, KAN, CAP, OFX, MXF, LZD, and CS is even better than with the 24 h method (data not shown). At times we observed differences in ΔCt of strains from experiment to experiment (e.g., H37Rv pre-treated with 72 hours of RIF yielded a ΔCt of approximately −5 in FIG. 1B versus−11 in 1D), which we presume relates to the growth state of the control Tb. However this is a factor with any phenotypic DST and the assay remained highly reproducible in our hands, with tight standard deviations in the ΔCt results (FIG. 4). Importantly, there were only seven (7/429=1.6%) instances where the D29 phage-qPCR assay was discrepant with the conventional methods. These were due to a capreomycin result near the cutoff and 6 ethionamide discrepancies, a drug where DST methods are known to be imperfect (16, 23).

The protocol is amenable to high throughput given the microplate format and can be performed by any laboratory currently performing culture-based DST that has access to a real-time PCR thermocycler. We hope this assay can be tested in other settings to refine the ΔCt cutoffs and examine additional isolates. The isolates used in these experiments were generally of high-level resistance, and thus the performance across a range of MICs needs to be further evaluated. Due to relatively few isolates resistant to moxifloxacin and other second line drugs, and none for linezolid and cycloserine, evaluation of the assay for these types of resistant strains was limited. In the future we aim to evaluate the performance of the assay on a larger variety of resistant isolates and adapt the assay for direct specimens.

Finally, the ΔCt value correlated with the MIC for most drugs. The role of MICs in the management of Tb is not yet defined, but we speculate it will find use in treating MDR and XDR Tb where drug options are limited. Even in Virginia we have noted that the majority Tb patients who are responding slowly to 4-drug therapy have C₂ᵢ serum levels below the expected range and often below the critical concentration for INH or RIF (13). In this context knowing the MIC of the organism may become clinically relevant, and we feel this ΔCt quantitative metric is strength of this assay.

The present application discloses results of experiments that were designed to develop a rapid PCR-based phenotypic drug susceptibility assay that utilizes amplification of D29 phage nucleic acid within about 24 hours of incubation with drug treated M. tuberculosis.

### Example 2

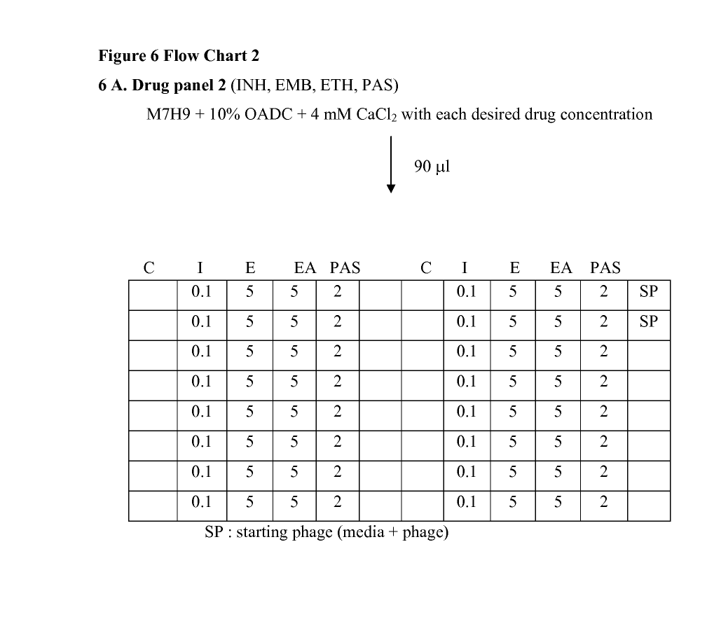

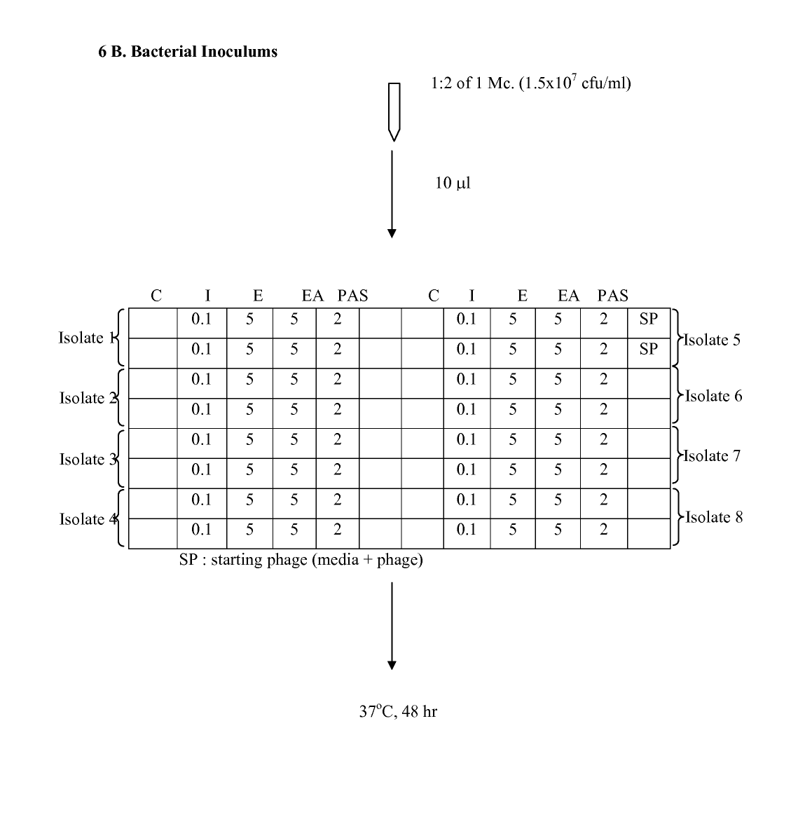

This example provides the experiments/assays in summary form which are also illustrated schematically in FIGS. 5 and 6.

1. Inoculum preparation
1.1 pick bacterial colony 1 loop full into glassbeed tube, vortex for 30 sec
1.2 add M7H9+10% OADC 2 ml, vortex for 30 sec let stand for 30 min
1.3 adjust turbidity in a new M7H9+10% OADC tube to 1.0 McFarland Standard (≈3×10⁷ cfu/ml)
1.4 dilute bacterial cell 1:2 in M7H9+10% OADC+4 mM CaCl₂ (≈1.5×10⁷ cfu/ml)

2. Drug susceptibility setting
For RIF, STR, AMK, KAN, CAP, OFX, MXF, LZD, CS
2.1 pipette medium with and without drug 90 μl into 96 well PCR plate
2.2 inoculate bacterial cell 10 μl (≈1.5×10⁵ cfu) into each well except starting phage control well (media without drug+phage)
2.3 add D29 phage (≈1.5×10⁵ pfu/ml) 10 μl into each well (≈1.5×10³ pfu)
2.4 cover plate with adhesive seal, incubate at 37° C. for 24 hr
For INH, EMB, ETH, PAS
2.5 pipette medium with and without drug 90 μl into 96 well PCR plate
2.6 inoculate bacterial cell 10 μl (≈1.5×10⁵ cfu) into each well except starting phage control well (media without drug+phage)
2.7 cover plate with adhesive seal, incubate at 37° C. for 48 hr
2.8 add D29 phage (≈1.5×10⁵ pfu/ml) 10 μl into each well (≈1.5×10³ pfu)
2.9 cover plate with adhesive seal, incubate at 37° C. for 24 hr
   (This procedure can be reduced in time).

3. DNA isolation
3.1 recover adhesive seal then cover plate with cap strip
3.2 incubate in 96 well heating block 100° C. for 30 min
3.3 centrifuge 4000 rpm for 15 min
3.4 pipette supernatant into a new PCR plate, store at −20° C. for DNA template

4. qPCR
4.1 amplify DNA sample using D29 phage specific primer using SYBR green detection system
4.2 analyze ΔCt (Ct control−Ct drug) compare with standard agar proportion result
4.3 plot scatter graph, calculate appropriate cut off for each drug using ROC analysis

Flow Charts 1 and 2 (FIGS. 5 and 6) show schematically some of the methods for using clinical test samples, growing them in the presence or absence of a drug, adding the D29 mycobacteriophage to infect the mycobacteria, and then sampling DNA and quantifying the phage DNA as a measure of the effect of the drug(s) on the bacteria. The Charts in FIGS. 5 and 6 demonstrate that different numbers of drugs can be tested and that the assay can be set up to easily test more than one drug. For example, FIG. 5 demonstrates the results testing nine drugs in an assay while FIG. 6 demonstrates the results of testing four drugs.

These results demonstrate that using the assay of the invention, drugs such as rifampin, streptomycin, amikacin, kanamycin, capreomycin, ofloxacin, moxifloxacin, linezolid, and cycloserine prevent phage replication in susceptible strains within 24 hours, while isoniazid, ethambutol, ethionamide, and p-aminosalicylic acid require 1-2 days preincubation to prevent phage growth.

Conclusions:

The D29 qPCR assay disclosed herein provides a rapid, molecular, phenotypic drug susceptibility assay for 13 first and second line antituberculosis drugs. Results are obtainable in 1-3 days and can triage rapid therapy of MDR patients while conventional DST is pending.

See Tables 1-3 (Table 3 is also referred to as Table 51).

TABLE 1
Critical concentrations for susceptibility testing
used for D29 phage-qPCR, agar proportion method,
and MIC range of Sensititre ® MYCOTB assay

| | | | Sensititre ® |
|---|---|---|---|
| | D29 phage-qPCR | M7H10 agar | MYCOTB |
| | (M7H9 Broth) | proportion | (M7H9 Broth) |
| | μg/ml | μg/ml | μg/ml |
| Antimicrobial agent | Critical concentration | | MIC range tested |
| Isoniazid | 0.1 | 0.2 | 0.03-4 |
| Rifampin | 1 | 1 | 0.12-16 |
| Ethambutol | 5 | 5 | 0.5-32 |
| Streptomycin | 1 | 2 | 0.25-32 |
| Amikacin | 1 | 5 | 0.12-16 |
| Kanamycin | 1 | 5 | 0.6-40 |
| Capreomycin | 2.5 | 10 | ND |
| Ofloxacin | 2 | 2 | 0.25-32 |
| Moxifloxacin | 0.25 | 2 | 0.06-8 |
| Ethionamide | 5 | 5 | 0.3-40 |
| para-aminosalicylic | 2 | 2 | 0.5-64 |
| acid | | | |
| Cycloserine | 30 | 30 | 2-256 |
| Linezolid | 1 | 1 | ND |

ND: Not done

TABLE 2
Accuracy of D29 phage-qPCR compared with agar proportion

| Antimicrobial | D29 phage-qPCR | Agar proportion (n) | | Accuracy |
|---|---|---|---|---|
| agent | ΔCt | Susceptible | Resistant | (%) |
| Isoniazid | <−6.0 | 10 | 0 | 100 |
| | ≧−6.0 | 0 | 23 | |
| Rifampin | <−4.0 | 11 | 0 | 100 |
| | ≧−4.0 | 0 | 22 | |
| Ethambutol | <−4.0 | 25 | 0 | 100 |
| | ≧−4.0 | 0 | 8 | |
| Streptomycin | <−3.0 | 13 | 0 | 100 |
| | ≧−3.0 | 0 | 20 | |
| Amikacin | <−4.0 | 31 | 0 | 100 |
| | ≧−4.0 | 0 | 2 | |
| Kanamycin | <−4.0 | 31 | 0 | 100 |
| | ≧−4.0 | 0 | 2 | |
| Capreomycin | <−6.0 | 30 | 0 | 97 |
| | ≧−6.0 | 1 | 2 | |
| Ofloxacin | <−4.0 | 30 | 0 | 100 |
| | ≧−4.0 | 0 | 3 | |
| Moxifloxacin | <+0.3 | 31 | 0 | 100 |
| | ≧+0.3 | 0 | 2 | |
| Ethionamide | <−5.0 | 21 | 2 | 82 |
| | ≧−5.0 | 4 | 6 | |
| para- | <−3.0 | 28 | 0 | 100 |
| aminosalicylic | ≧−3.0 | 0 | 5 | |
| acid | | | | |
| Cycloserine | <−2.0* | 33 | 0 | 100 |
| | ≧−2.0 | 0 | 0 | |
| Linezolid | <−3.0* | 33 | 0 | 100 |
| | ≧−3.0 | 0 | 0 | |

*cutoffs for cycloserine and linezolid cannot be reliably ascribed due to the lack of resistant strains.

TABLE S1 (3)
Correlation among drug resistant associated gene mutation,
standard agar proportion, and D29-qPCR assay

| | | | Agar proportion | | D29 phage assay | |
|---|---|---|---|---|---|---|
| | | | Resistant | | Resistant | |
| Drugs | Locus | Mutation | (MIC*) | susceptible | (ΔCt) | susceptible |
| INH | inhA | C(−15)T | 2(2, 4) | 0 | 2(0.1, 2.3) | 0 |
| | katG | Ser315Thr | 19(1-4) | 0 | 19(−1.2-2.5) | 0 |
| | inhA | T(−8)C/Ser315Thr | 1(4) | 0 | 1(4.9) | 0 |
| | and | | | | | |
| | katG | | | | | |
| | | no mutation | 1(2) | 10 | 1(0.9) | 10 |
| RIF | rpoB | Leu511Pro | 1(2) | 0 | 1(−0.4) | 0 |
| | | Asp516Val | 2(8, 16) | 0 | 2(−3.4, −0.8) | 0 |
| | | His526Tyr | 2(16) | 0 | 2(1.1, 1.8) | 0 |
| | | Ser531Leu | 17(16) | 0 | 17(−1.7-3.6) | 0 |
| | | no mutation | 0 | 11 | 0 | 11 |
| EMB | embB** | Met306Val | 4(4-8) | 0 | 4(−0.4-1.3) | 0 |
| | | Met306Ile | 1(4) | 1 | 1(−1.9) | 1 |
| | | Met306Val/Glu378Ala | 1(8) | 0 | 1(−2.3) | 0 |
| | | Asp328Tyr | 1(4) | 0 | 1(−1.5) | 0 |
| | | Glu378Ala | 0 | 6 | 0 | 6 |
| | | Pro404Ser | 1(4) | 1 | 1(−1.1) | 1 |
| | | Gly406Asp | 0 | 5 | 0 | 5 |
| | | no mutation | 0 | 12 | 0 | 12 |
| AMK | rrs | A1401G | 1(16) | 0 | 1(−2.9) | 0 |
| | | G1484T | 1(8) | 0 | 1(2.4) | 0 |
| | | no mutation | 0 | 31 | 0 | 31 |
| KAN | rrs | A1401G | 1(40) | 0 | 1(−3.5) | 0 |
| | | G1484T | 1(40) | 0 | 1(0.6) | 0 |
| | | no mutation | 0 | 31 | 0 | 31 |
| | eis | no mutation | 2 | 31 | 2 | 31 |
| CAP | rrs | A1401G | 1(ND) | 0 | 1(−5.4) | 0 |
| | | G1484T | 1(ND) | 0 | 1(−3.3) | 0 |
| | | no mutation | 0 | 31 | 1(−5.4) | 30 |
| OFX | gyrA | Asp94Gly | 3(8-16) | 0 | 3(−2.8-2.7) | 0 |
| | | no mutation | 0 | 30 | 0 | 30 |
| MXF | gyrA | Asp94Gly | 2(4) | 1(2) | 2(2, 2.2) | 1(−1.4) |
| | | no mutation | 0 | 30 | 0 | 30 |

*MIC in Trek Sensititre ® MYCOTB assay,
**6 embB Glu378Ala mutations were detected and not considered resistance mutations as per Campbell et al (8).

### Example 3

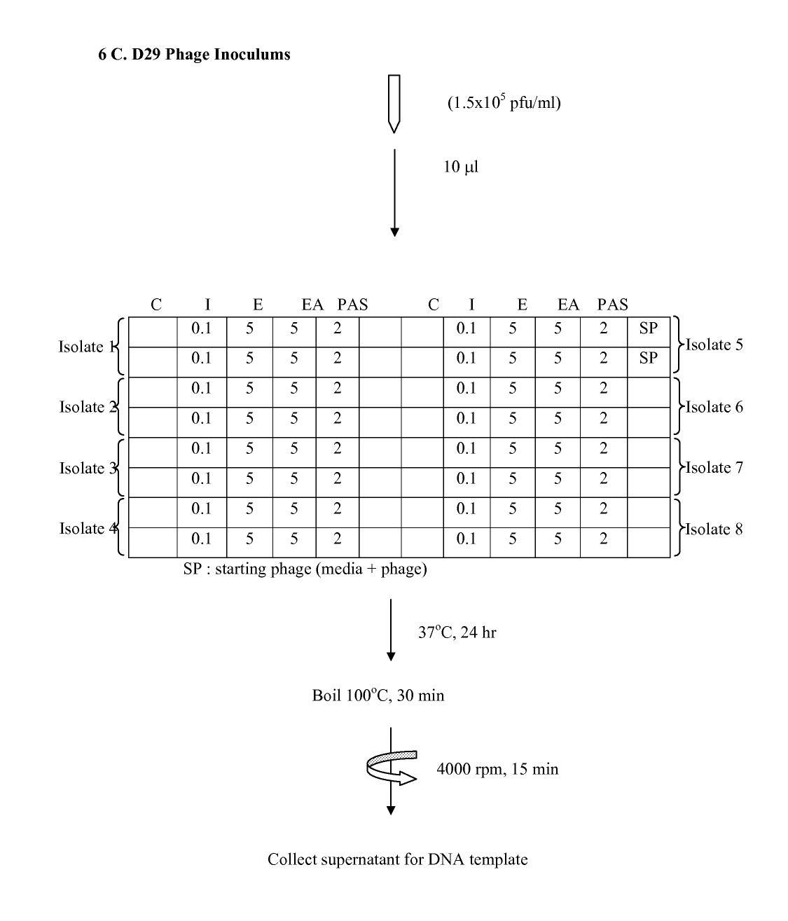

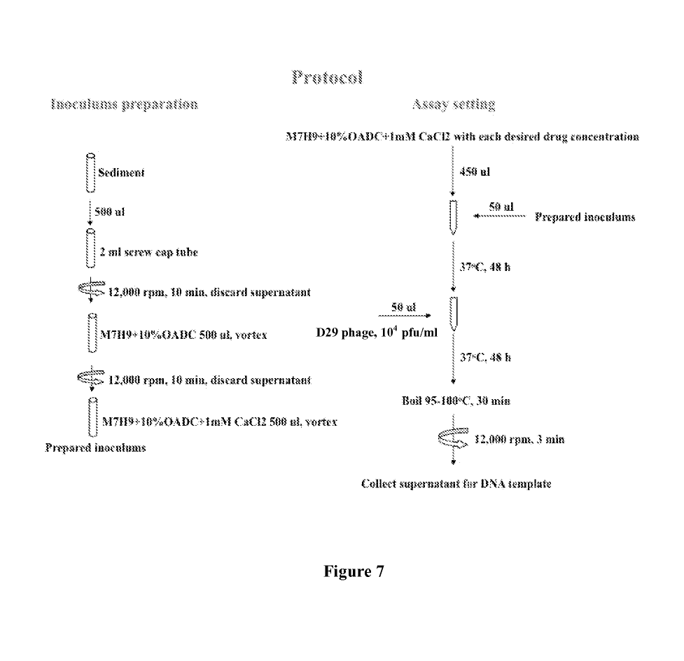

Next, we pursued the assay on sputum (direct specimen sediments). Briefly, we have observed the following: 48 h of phage incubation works better than 24 h, with no further improvement at 72 h. Optimal starting phage is 10³ pfu/ml. There was no difference in shaking and not shaking for the phage incubation step. Thus, the final protocol is found in FIG. 7.

We have processed this on 207 smear positive sediments.

Provided are the results on 22 of the sediments which amplified above background (these results obtained within 96 h of sputum collection), which yielded good accuracy versus culture/agar proportion results (these results came 2 months later). See Table 4.

TABLE 4
Accuracy of Assay Using Sputum Samples

| | |
|---|---|
| Sediment | Culture |

| samples | Total 22 | S | R | Accuracy (%) |
|---|---|---|---|---|
| INH | S | 18 | 0 | 86.4 |
| | R | 3 | 1 | |
| RIF | S | 21 | 0 | 100 |
| | R | 0 | 1 | |
| STR | S | 20 | 1 | 95.5 |
| | R | 0 | 1 | |
| EMB | S | 22 | 0 | 100 |
| | R | 0 | 0 | |

Thus the assay is accurate on sputum so long as detection of phage is above background.

### Example 4

In another set of experiments, about 82% (208/255) of isolates yield good phage amplification above background (defined as ΔCt of 3 above starting phage) but 18% do not. Most of these latter isolates also did not grow via agar proportion (30/47=64%), thus they are poor growers, and are often MDR strains. Moreover, the number of isolates that fail by phage is less than the number that fail by agar proportion. Other reasons for this could be that some Tb strains are resistant to phage infection, they grow better on L-J (where they are starting from) vs. 7H10, etc. We are evaluating changes in CaCl2 concentration (1 vs. 2 vs. 4 mM) but preliminarily this yields no difference. We are also determining whether our working cutoff of needing a ΔCt of 3 above starting phage is too stringent. The most likely fix would be to increase the time in 7H9 prior to addition to phage. In any event, with this greater number of isolates we were better able determine accuracy, which looks excellent. For example for first line drugs, see Table 5.

TABLE 5

Agar proportion

| | | | |
|---|---|---|---|
| Total 154 isolates | S | R | Accuracy (%) |

| | | | | |
|---|---|---|---|---|
| INH | S | 118 | 5 | 90.91 |
| | R | 9 | 22 | |
| RIF | S | 142 | 5 | 95.45 |
| | R | 2 | 5 | |
| STR | S | 135 | 3 | 98.05 |
| | R | 0 | 16 | |
| EMB | S | 145 | 8 | 94.16 |
| | R | 1 | 0 | |

We were also able to better refine the ΔCt cutoffs that yield greater accuracy for susceptible/resistant (below). For drugs where two ΔCt are shown the lower one is the original one as published (JCM) while the upper one is the new improved ΔCt cutoff.

TABLE 6

| Second line | | % Accuracy |
|---|---|---|
| Drugs | Cut-off | 64 isolates |
| AMK | −3 | 96.9 |
| | −4 | 93.8 |
| KAN | −4 | 93.8 |
| CAP | −6 | 90.6 |
| OFX | −4 | 93.8 |
| MXF | −2 | 95.3 |
| | 0.3 | 95.3 |
| LZD | −2 | 100 |
| | −3 | 96.9 |
| ETH | −3 | 88.4 |
| | −5 | 75.0 |
| PaS | −3 | 89.1 |
| CS | −2 | 100 |
| | −3 | 95.3 |

Most of these data were generated using susceptible isolates (particularly for the second line drugs) so we still need to evaluate on greater numbers of resistant isolates.

Next, we pursued the assay on sputum (direct specimen sediments). Briefly, we have observed the following: 48 h of phage incubation works better than 24 h, with no further improvement at 72 h. Optimal starting phage is 10̂3 pfu/ml. There was no difference in shaking and not shaking for the phage incubation step. Thus the final protocol can be found in FIG. 7.

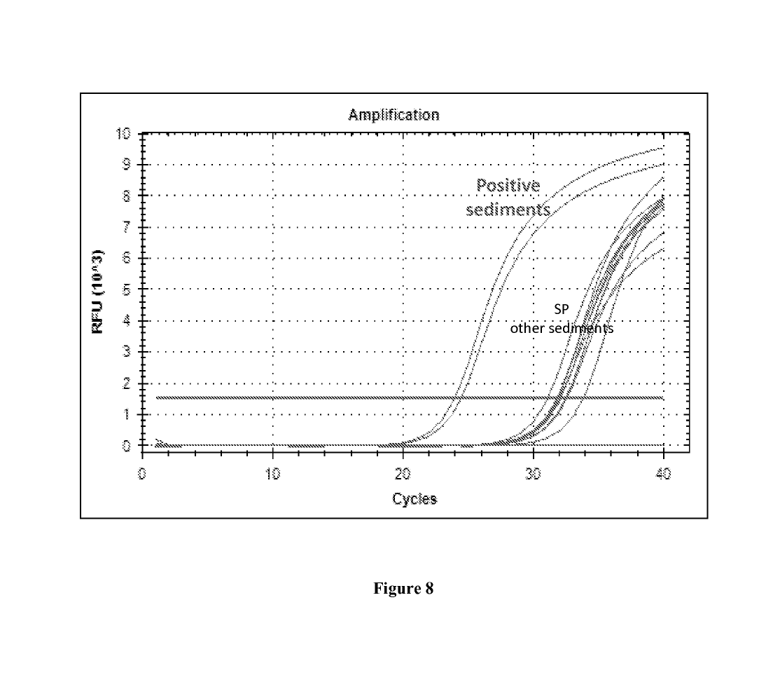

We have processed this on 207 smear positive sediments. Of these, 54 yielded phage amplification above starting phage (ΔCt of 3). However, upon repeating the phage assay in order to set it up with drugs, only 22 of these 54 yielded phage amplification again above starting phage. See FIG. 8.

Thus the assay is accurate on sputum, but the limitation is in the number of specimens that repeatably yield phage amplification above background (22/207=11%). However these sediments were not all fresh, thus we are awaiting new results. We would expect improvement with addition of more time in 7H9 prior to phage (e.g., 96 h vs. 48 h).

### Other Work Ongoing:

MGIT broths. We are evaluating the assay in BACTEC MGIT broths, which are frequently contaminated with bacteria, since the phage assay should still work in that setting (without the time required for subculturing to pure Tb).
96 well plate format. We have since made additional improvements to the SOP to make it faster to set up and less manipulations, including converting it to a 96 well plate format. Using deep well 96 well plates the Tb/drug/phage can be incubated and then heated, centrifuged and the supernatant multichannel-pipetted directly to a parallel 96 well plate for PCR (the deep well plates are useful because it allows pelleting of the proteinaceous media).
We are reevaluating the ΔCt cutoff. Previously we have used strongly growing isolates and have used the ΔCt from Control Tb to Tb+drug (this tells how well inhibited the Tb is with drug). We may wish to examine ΔCt from starting phage to Tb+drug (this tells how viable the Tb is with drug).
Adding a precise quantity of Starting Phage is a critical step in the assay. Thus we are evaluating the stability of phage under storage. When new stock Phage is prepared we are implementing a more detailed QC protocol to include a standard curve (dilution series) run to correlate with the pfu/ml quantitation.
TaqMan probe. We are evaluating a TaqMan probe assay for more specificity. Preliminarily this shows less sensitivity, in that starting phage is detected at around 40-42 cycles, so we need to run the assay for 45 cycles. Work is ongoing.
Bangladesh has undergone training and has data on ˜16 isolates, with comparison L-J and using TREK plates. qPCR done on BioRad CFX. Again a minority of isolates does not support phage growth and these are often MDR. Awaiting final analysis, but accuracy of phage assay appears good vs. L-J or TREK.
Tanzania has undergone training and has complete data on 21 isolates, 7 susceptible strains and 14 MDR strains. Again, awaiting final analysis, but accuracy of phage assay appears good vs. MGIT or TREK. Discrepant results tend to be near the MIC breakpoints (within one dilution) or near the phage ΔCt cutoffs.

### Example 5

### Standard Operating Procedure

### Principle

This protocol describes a 3-day assay to evaluate the drug resistance of M. tuberculosis to first and second-line drugs using real-time PCR of mycobacteriophage D29 DNA. M. tuberculosis is cultured with and without drug followed by incubation with D29 mycobacteriophage and then real-time PCR. Mycobacteriophage D29 infects and replicates in viable bacterial cells. The change in phage DNA real-time PCR cycle threshold (C_{T}) between control M. tuberculosis and M. tuberculosis treated with drugs is calculated to correlate with standard drug susceptibility results. See media preparation, stock antimicrobial preparation, and D29 phage stock preparation protocols below.

### Inoculum Preparation: Day 0

1. Use 2-3 week old growth from L-J or 7H10.QC strain H37Rv is tested each run.
2. Place 5-6 sterile 5 mm glass bead in to a 14 ml plastic tube
3. Add one—2 drops 7H9/OADC
4. Scrape 1 loop full of colonies and place into glass bead tube, emulsify by vortexing for 30 sec
5. Use 2-3 week old growth from L-J or 7H10.QC strain H37Rv is tested each run.
6. Place 5-6 sterile 5 mm glass bead in to a 14 ml plastic tube
7. Add one—2 drops 7H9/OADC
8. Scrape 1 loop full of colonies and place into glass bead tube, emulsify by vortexing for 30 sec
9. Pipet 2 ml M7H9/OADC into the same tube, vortex for 30 sec
10. Let settle at room temperature for 15-30 min
11. In a new tube pipet 3 ml 7H9/OADC. Use the top ⅓ of the inoculum supernatant and adjust the turbidity to 0.5 McFarland Standard—usually 6-10 drops is sufficient. (˜1.5×10⁷ cfu/ml)
12. Further dilute 1:10: 100 ul bacteria suspension.:900 ul M7H9/OADC+1 mM CaCl₂: (≈1.5×10⁶ cfu/ml). This is the final inoculum.

### Isolate+Drug Assay Set Up: Day 0

1. For each isolate label 13 tubes with isolate and drug; plus one control tube (media+isolate without drug). There are 13 drugs in the current protocol—total tubes=14 per isolate
2. Pipet 450 ul of each drug (assay concentration) into their respective tubes
3. Pipet 450 ul of 7H9/OADC/1 mM CaCl₂ into each isolate's Control Tube
4. Pipet 50 ul of prepared inoculum into each tube
5. Each run: include two tubes with Media Only (no drug, no isolate)
6. Incubate 35-37° C. for 36-48 hours

### Phage Addition: Day 2

1. Add 50 ul D29 Phage (assay concentration) to each of the 14 tubes/isolate (˜1.5×10⁴⁾)
2. Add 50 ul D29 Phage to one of the Media Only Control tubes. This is the “Starting Phage” control tube, the remaining “Media Only” control tubes is the Negative Control (no drug, no isolate, no D29 Phage)
3. Incubate 35-37° C. for 18-24 hours

### DNA Extraction: Day 3

1. Heat tubes 100° C. for 30 minutes
2. Centrifuge 12000 rpm for 3 minutes
3. The supernatant is the DNA template
4. Store at −20° C. or proceed to PCR Assay

### Real-Time PCR

Amplify DNA sample using D29 phage specific primer using SYBR green detection system Forward primer: 5′-GCCACCAGGAGCCACGAAC-3′ (SEQ ID NO: 3) Reverse primer: 5′-AATAGGGAAGGAGTCTGCGTTTG-3′ (SEQ ID NO. 4) Product size 139 bp; Tm 86° C.

### Notes for PCR Practices

1. Maintain separate areas for sample preparation, PCR setup, and amplification.
2. Never bring amplified PCR products into the PCR setup area.
3. Use aerosol barrier pipette tips.
4. When preparing master mixes wear a clean lab coat (not previously worn while handling amplified PCR products or used during sample preparation).
5. Change gloves whenever you suspect that they are contaminated.
6. Centrifuge tubes before opening.
7. Keep reactions and components capped as much as possible.
8. Clean lab benches and equipment with 10% bleach solution, and Eliminase or DNAzap

### Assemble PCRs

1. Prepare Master Mix
2. Work in the DNA free room
3. Thaw master mix components keep enzyme mix in a bench top tube cooler if possible
4. Determine the number of reactions and prepare 10% extra master mix in a microcentrifuge tube
5. Mix master mix then quick spin to remove any drops from the lid.
6. Pipet 20 ul master mix into each well. Cover the plate and exit the DNA free room

TABLE 7

| Component | 1 rxn | 20 rxn | 50 rxn | 100 rxn |
|---|---|---|---|---|
| NFW | 7 ul | 140 ul | 350 ul | 700 ul |
| BioRad SYBR green | 12.5 ul | 250 ul | 625 ul | 1250 ul |
| master mix (2x) | | | | |
| Forward primer (50 uM) | 0.25 ul | 5 ul | 12.5 ul | 25 ul |
| Reverse primer (50 uM) | 0.25 ul | 5 ul | 12.5 ul | 25 ul |
| MM total | 20 ul | | | |
| DNA template | 5 ul | | | |
| Total | 25 ul | | | |

1. Add template DNA: pipet 5 ul sample DNA into each reaction
2. NTCs: Include at least 2 NTCs on each plate: pipet 5 ul NFW
3. Seal the PCR plate, Quick spin the plate
4. Load the plate into the thermocycler

### PCR Cycle Program

1. Initial denaturation 95° C. 5 min

| | | | |
|---|---|---|---|
| 1. | Initial denaturation 95° C. 5 min | | |
| 2. | Denaturation 94° C. 10 sec | | |
| | | {close oversize brace} | 40 cycles |
| 3. | Annealing 65° C. 20 sec | | |
| 4. | Extension 72° C. 15 sec, plate read | | |
| 5. | Melt curve analysis 15 sec | | |
| | 72° C.-95° C., 0.5° C. increment, | | |
| 6. | Hold 4° C. | | |

4. Extension 72° C. 15 sec, plate read
5. Melt curve analysis 15 sec 72° C.-95° C., 0.5° C. increment,
6. Hold 4° C.

### Analysis

### Notes:

A. If the Ct for the isolate's control tube is not earlier then the “starting phage” tube, the results for that isolate are invalid. As this would indicate, the phage did not replicate in that isolate.
B. Every reaction except the “media only” negative control and the NTCs should show amplification.
   4.4 Export data file to excel
   4.5 Calculate A Ct (Ct control−Ct drug)
   4.6 Plot scatter graph, calculate appropriate cut off for each drug using ROC analysis
   4.7 Compare with standard agar proportion. MGIT DST, and or TREK MIC results

### Interpretation

Δ Cts values corresponding to “resistant”, “susceptible” or MICs (ug/ml) by conventional methods have not been definitively assigned,

TABLE 8

| Supplies | Vendor | Cat. Number |
|---|---|---|
| Isoniazid | Sigma- | I3377-5G |
| | Aldrich | |
| Rifampicin | Sigma- | R3501-1G |
| | Aldrich | |
| Streptomycin sulfate | Sigma- | S6501-5G |
| | Aldrich | |
| Kanamycin sulfate | Sigma- | K4000-1G |
| | Aldrich | |
| Ofloxacin | Sigma- | O8757-1G |
| | Aldrich | |
| Ethionamide | Sigma- | E6005-5G |
| | Aldrich | |
| Para-aminosalicylic acid | Sigma- | A79604-5G |
| | Aldrich | |
| D-cycloserine | Sigma- | C6880-1G |
| | Aldrich | |
| Calcium chloride dihydrate (CaCl₂.2H₂O) | Sigma- | C3881-500G |
| | Aldrich | |
| Ethambutol hydrochloride | MP | 157949 |
| | Biomedicals | |
| Amikacin | MP | 150342 |
| | Biomedicals | |
| Capreomycin sulfate | MP | 154924 |
| | Biomedicals | |
| Moxifloxacin | UVA | --------------- |
| | Pharmacy | |
| Linazolid | UVA | --------------- |
| | Pharmacy | |
| Difco Middlebrook7H9 Broth | Fisher | DF0713-17-9 |
| Difco Middlebrook7H10 agar | Fisher | DF0627-17-4 |
| Middlebrook OADC Enrichment | Fisher | B12351 |
| Glass Beads 5 mm | Fisher | CG110104 |
| Culture test tube (14 ml) | Fisher | --------------- |
| 10 μl Long Reach Bather Tips | VWR | 87001-688 |
| 200 μl Barrier Tips | | ---------------- |
| 1000 μl Barrier Tips | | ---------------- |
| Loops | Fisher | |
| 2 ml screw cap micro tubes | SARSTEDT | 72694406 |
| D29_PP-F primer | Operon | |
| D29_PP-R primer | Operon | |
| iQ SYBR Green Super mix | Bio-Rad | 1708882 |
| PCR Plates and Seals | Bio-Rad | MSB1001 |
| Equipment | | |
| vortex | | |
| heat block 100 C. | | |
| microcentrifuge | | |
| Pipetors P10-P1000 | | |
| Incubator 35-37° C. | | |
| Real time thermocycler | | |
| Primer sequences March 2012 | | |
| Forward primer: 5′- | | |
| GCCACCAGGAGCCACGAAC-3′ | | |
| (SEQ ID NO 3) | | |
| Reverse primer: 5′- | | |
| AATAGGGAAGGAGTCTGCGTTTG-3′ | | |
| (SEQ ID NO 4) | | |

### Media Preparation

### Middlebrook 7H10 Agar

Weigh 19 g of M7H10 medium (Becton Dickinson and company, Sparks, Md., USA) powder, place into a 1 L bottle, add 900 ml of distilled water, add 5 ml of glycerol). Autoclave at 121° C. for 15 minutes, Cool to 56° C. Aseptically add 100 ml of Middlebrook OADC (oleic acid, albumin, dextrose, catalase).

Pour 30 ml/plate or 40 ml into a 50 ml tube, allow to harden.
Store at 4° C. for up to 3 months
100 mM CaCl₂ Weigh CaCl₂.2H₂O (dihydrate) 1.47 g add 100 ml distilled water, autoclave at 121° C. for 15 min.
Store at 4° C. for up to 6 months.
Middlebrook 7H9 broth/OADC
Weigh 4.7 g of M7H9 broth (Becton Dickinson and company, Sparks, Md., USA) powder, place into a 1 L bottle, add 900 ml of distilled water, add 2 ml of glycerol).
Autoclave at 121° C. for 15 minutes, cool to 56° C. Aseptically add 100 ml of Middlebrook OADC.
Store at 4° C. for up to 3 months.
Middlebrook 7H9 Broth with Calcium: “7H9/OADC+1 mM CaCl₂”
Note: 1 mM CaCl₂ is required whenever 7H9 is used with D29 Phage
Add 1 ml 100 mM CaCl₂ to 99 ml prepared 7H9 broth (7H9+OADC)
Store at 4° C. for up to 3 months

### Antimicrobial Agent Preparation

Prepare Stock once each year
Prepare “Assay Concentration” each month
Stock Concentration Preparation (10,000 μg/ml)
1. Dissolve antimicrobial agents in the appropriate solvents to obtain a stock concentration of 10,000 ug/ml and aliquot 500 μl in sterile 1.5 ml tube
2. Store at −70° C. for up to one year. Thaw each aliquot only once and discard any remaining in the tube. Do not refreeze stock concentrations once thawed.

### Working Dilution and Assay Concentration (Prepare Monthly)

1. Thaw aliquot of stock drug.
2. Dilute stock drug 1:10 in sterile distilled water (SDW)=100 ul stock+900 ul SDW. This is the “working dilution”
3. Aliquot 50 ml 7H9/OADC+1 mM CaCl₂ into 50 ml conical tubes (one tube for each drug)
4. Refer to Table below and add x ul “working dilution” (prepared in step 2 above into the 50 ml tubes. This is the “Assay Concentration”
5. Store “Assay Concentration” at 4 C up to 1 month, protect from light by wrapping tubes in foil.
6. Discard remaining ‘working dilution”

TABLE 9

| | | | | Volume of drug | |
|---|---|---|---|---|---|
| | | | Dilute Stock | to add to 50 ml | |
| Antimicrobial | | Stock | 1:10 | 7H9/OADC + 1 mM | Final |
| agent | Solvent | ug/ml | Drug:SDW | CaCl₂ | ug/ml |
| Isoniazid | SDW | 10000 | 100 ul + 900 ul | 5 μl | 0.1 |
| Rifampicin | DMSO | 10000 | 100 + 900 | 50 μl | 1 |
| Streptomycin | SDW | 10000 | 100 + 900 | 50 μl | 1 |
| Ethambutol | SDW | 10000 | 100 + 900 | 250 μl | 5 |
| Amikacin | SDW | 10000 | 100 + 900 | 50 μl | 1 |
| Kanamycin | SDW | 10000 | 100 + 900 | 50 μl | 1 |
| Capreomycin | SDW | 10000 | 100 + 900 | 125 μl | 2.5 |
| Ofloxacin | 0.1N | 10000 | 100 + 900 | 100 μl | 2 |
| | NaOH | | | | |
| Moxifloxacin | None | 1600 | — | 7.81 μl | 0.25 |
| Linezolid | None | 2000 | — | 25 μl | 1 |
| Ethionamide | DMSO | 10000 | 100 + 900 | 250 μl | 5 |
| p-aminosalicylic | Ethanol | 10000 | 100 + 900 | 100 μl | 2 |
| acid | | | | | |
| Cycloserine | SDW | 10000 | 100 + 900 | 1500 μl | 30 |

### D 29 Phage Preparation:

Prepare fresh every 6 months. Store at 4° C.

### A. Phage Propagation:

1. Prepare M. smegmatis Culture
   A. Inoculate M. smegmatis into ˜100 ml 7H9/OADC
   B. Incubate 35-37° C. for 48-72 hours (stationary phase)
2. Melt 40 ml 7H10 agar, cool to 56° C.
3. Add 5 ml OADC
4. Add 5 ml M. smegmatis (stationary phase)
5. Pour into 2 petri plates, allow to harden
6. Prepare a 1:10 dilution of the 10⁴ working phage stock) 100 ul phage+900 ul 7H09/OADC/1 mM CaCl₂
7. Spread 100 ul D29 Phage onto surface agar
8. Incubate 35-37° C. for 18-48 hours
9. Examine bacterial growth for plaques: a large number should be visible (>100/plate).
10. Pipet 10 ml 7H09/OADC/1 mM CaCl₂ on to the agar surface
11. Incubate overnight 35-37 C
12. Remove liquid from surface and filter sterilize using 0.45 um syringe filter. This is the stock (high titer phage)
13. Store 4 C up to 6 months

B. Phage Quantitation:
1. Prepare M. smegmatis Culture (can use the same M. smegmatis culture batch used for propagation) or Inoculate M. smegmatis into ˜100 ml 7H9/OADC and Incubate 35-37° C. for 48-72 hours (stationary phase)
2. Prepare 7H10 agar plate for bottom layer of 2 layer plate Melt 40 ml 7H10, cool to 56 C, pour plates allow to harden.
3. Prepare D29 1:10 serial dilutions of stock D29 (high titer phage) Prepare 10 tubes for serial dilution by pipetting 900 ul 7H09/OADC/1 mM CaCl₂ into 10 tubes,
   A. Pipet 100 ul of stock high titer phage in to the first tube, mix, and continue to serial dilute 1:10 for the remaining 9 tubes.
4. Prepare overlay 7H10 agar/M. smegmatis/D29 layer in a second set of 10 tubes:
   A. Combine 200 ul M. smegmatis culture+10 ul each dilution phage in a 15 ml tube;
   B. Let stand 10 minutes
   C. Add 4 ml 7H10 agar to each M.smeg/phage dilution, mix
   D. Layer onto 7H10 plate, allow to harden.
   E. Incubate 35-37° C. for 24-48 hours
5. Count the plaque forming units pfu/plate:
   A. Choose the dilution plate where the plaques number 30-300 and count the plaques.
   B. Calculate the pfu/ml in the high titer stock

C. Phage Woring Concentration:
1. Dilute the high titer stock with 7H09/OADC/1 mM CaCl₂ to obtain a working D29 Phage concentration of 10⁴/ml. This is the phage preparation used in the assay set up. “Assay Concentration”
2. Store at 4° C. for up to 6 months
   Preparation of Inoculum from Other Colonies

Inoculum Preparation from MGIT Tube Positive
1. Pipette 500 μl MGIT+liquid into 2 ml screw cap tube
2. Centrifuge 12,000 rpm 10 min, discard supernatant
3. Add 500 μl M7H9+OADC, to pellet, vortex
4. Centrifuge 12,000 rpm 10 min, discard supernatant
5. Re-suspend pellet with 500 μl 7H9/OADC+1 mM CaCl2

Inoculum Preparation from Sputum Digest Sediment
1. Pipette 500 μl sediment into 2 ml screw cap tube
2. Centrifuge 12,000 rpm 10 min, discard supernatant
3. Add 500 μl M7H9+10% OADC, vortex
4. Centrifuge 12,000 rpm 10 min, discard supernatant
5. Re-suspend pellet with 500 μl 7H9/OADC+1 mM CaCl2

### BIBLIOGRAPHY

1. Ardito, F., B. Posteraro, M. Sanguinetti, S. Zanetti, and G. Fadda. 2001. Evaluation of BACTEC Mycobacteria Growth Indicator Tube (MGIT 960) automated system for drug susceptibility testing of Mycobacterium tuberculosis. J Clin Microbiol 39:4440-4444.
2. Banaiee, N., M. Bobadilla-del-Valle, P. F. Riska, S. Bardarov, Jr., P. M. Small, A. Ponce-de-Leon, W. R. Jacobs, Jr., G. F. Hatfull, and J. Sifuentes-Osornio. 2003. Rapid identification and susceptibility testing of Mycobacterium tuberculosis from MGIT cultures with luciferase reporter mycobacteriophages. J Med Microbiol 52:557-561.
3. Banaiee, N., V. January, C. Barthus, M. Lambrick, D. Roditi, M. A. Behr, W. R. Jacobs, Jr., and L. M. Steyn. 2008. Evaluation of a semi-automated reporter phage assay for susceptibility testing of Mycobacterium tuberculosis isolates in South Africa. Tuberculosis (Edinb) 88:64-68.
4. Bardarov, S., Jr., H. Dou, K. Eisenach, N. Banaiee, S. Ya, J. Chan, W. R. Jacobs, Jr., and P. F. Riska. 2003. Detection and drug-susceptibility testing of M. tuberculosis from sputum samples using luciferase reporter phage: comparison with the Mycobacteria Growth Indicator Tube (MGIT) system. Diagn Microbiol Infect Dis 45:53-61.
5. Bowman, B. U. J. 1968. Quantitative studies on some mycobacterial phage-host systems. J. Bacteriol 76:52-62.
6. Bruning, J. B., A. C. Murillo, O. Chacon, R. G. Barletta, and J. C. Sacchettini. 2011. Structure of the Mycobacterium tuberculosis D-alanine:D-alanine ligase, a target of the antituberculosis drug D-cycloserine. Antimicrob Agents Chemother 55:291-301.
7. Campbell, E. A., N. Korzheva, A. Mustaev, K. Murakami, S. Nair, A. Goldfarb, and S. A. Darst. 2001. Structural mechanism for rifampicin inhibition of bacterial ma polymerase. Cell 104:901-912.
8. Campbell, P. J., G. P. Morlock, R. D. Sikes, T. L. Dalton, B. Metchock, A. M. Starks, D. P. Hooks, L. S. Cowan, B. B. Plikaytis, and J. E. Posey. 2011. Molecular detection of mutations associated with first- and second-line drug resistance compared with conventional drug susceptibility testing of Mycobacterium tuberculosis. Antimicrob Agents Chemother 55:2032-2041.
9. David, H. L., S. Clavel, and F. Clement. 1980. Absorption and growth of the bacteirphage D29 in selected mycobacter. Ann Virol 131:167-184.
10. Deng, L., K. Mikusova, K. G. Robuck, M. Scherman, P. J. Brennan, and M. R. McNeil. 1995. Recognition of multiple effects of ethambutol on metabolism of mycobacterial cell envelope. Antimicrob Agents Chemother 39:694-701.
11. Foongladda, S., S. Pholwat, B. Eampokalap, P. Kiratisin, and R. Sutthent. 2009. Multi-probe real-time PCR identification of common Mycobacterium species in blood culture broth. J Mol Diagn 11:42-48.
12. Gandhi, N. R., P. Nunn, K. Dheda, H. S. Schaaf, M. Zignol, D. van Soolingen, P. Jensen, and J. Bayona. 2010. Multidrug-resistant and extensively drug-resistant tuberculosis: a threat to global control of tuberculosis. Lancet 375:1830-1843.
13. Heysell, S. K., J. L. Moore, S. J. Keller, and E. R. Houpt. 2010. Therapeutic drug monitoring for slow response to tuberculosis treatment in a state control program, Virginia, USA. Emerg Infect Dis 16:1546-1553.
14. Jacobs, W. R., Jr., R. G. Barletta, R. Udani, J. Chan, G. Kalkut, G. Sosne, T. Kieser, G. J. Sarkis, G. F. Hatfull, and B. R. Bloom. 1993. Rapid assessment of drug susceptibilities of Mycobacterium tuberculosis by means of luciferase reporter phages. Science 260:819-822.
15. Kisa, O., A. Albay, O. Bedir, O. Baylan, and L. Doganci. 2003. Evaluation of FASTPlaqueTB-RIF for determination of rifampicin resistance in Mycobacterium tuberculosis complex isolates. Int J Tuberc Lung Dis 7:284-288.
16. Lin, S. Y., E. Desmond, D. Bonato, W. Gross, and S. Siddiqi. 2009. Multicenter evaluation of Bactec MGIT 960 system for second-line drug susceptibility testing of Mycobacterium tuberculosis complex. J Clin Microbiol 47:3630-3634.
17. Marei, A. M., E. M. El-Behedy, H. A. Mohtady, and A. F. Afify. 2003. Evaluation of a rapid bacteriophage-based method for the detection of Mycobacterium tuberculosis in clinical samples. J Med Microbiol 52:331-335.
18. McNerney, R. 2001. Phage replication technology for diagnosis and drug susceptibility testing. Methods Mol Med 54:145-154.
19. McNerney, R., B. S. Kambashi, J. Kinkese, R. Tembew, and P. Godfrey-Fausett. 2004. Development of a Bacteriophage Replication assay for Diagnosis of Pulmonary Tuberculosis. J Clin Microbiol 42:2115-2120.
20. NCCLS/CLSI. 2009. Susceptibility testing of Mycobacteria, Nocardiae, and other aerobic Actinomycetes; approved standard, vol. 20. NCCLS/CLSI.
21. Pai, M., S. Kalantri, L. Pascopella, L. W. Riley, and A. L. Reingold. 2005. Bacteriophage-based assays for the rapid detection of rifampicin resistance in Mycobacterium tuberculosis: a meta-analysis. J Infect 51:175-187.
22. Pearson, R. E., S. Jurgensen, G. J. Sarkis, G. F. Hatfull, and W. R. J. Jacobs. 1996. Construction of D29 shuttle plasmids and lucerferase resporter phages for detection of mycobacteria. Gene 183:129-136.
23. Pfyffer, G. E., D. A. Bonato, A. Ebrahimzadeh, W. Gross, J. Hotaling, J. Kornblum, A. Laszlo, G. Roberts, M. Salfinger, F. Wittwer, and S. Siddiqi. 1999. Multicenter laboratory validation of susceptibility testing of Mycobacterium tuberculosis against classical second-line and newer antimicrobial drugs by using the radiometric BACTEC 460 technique and the proportion method with solid media. J Clin Microbiol 37:3179-3186.
24. Pholwat, S., S. Heysell, S. Stroup, S. Foongladda, and E. Houpt. 2011. Rapid first- and second-line drug susceptibility assay for Mycobacterium tuberculosis isolates by use of quantitative PCR. J Clin Microbiol 49:69-75.
25. Rengarajan, J., C. M. Sassetti, V. Naroditskaya, A. Sloutsky, B. R. Bloom, and E. J. Rubin. 2004. The folate pathway is a target for resistance to the drug para-aminosalicylic acid (PAS) in mycobacteria. Mol Microbiol 53:275-282.
26. Rondon, L., M. Piuri, W. R. Jacobs, Jr., J. de Waard, G. F. Hatfull, and H. E. Takiff. 2011. Evaluation of Fluoromycobacteriophages for Detecting Drug Resistance in Mycobacterium tuberculosis. J Clin Microbiol 49:1838-1842.
27. Sellers, M. I., W. L. Baxter, and H. R. Runnals. 1962. Growth Characteristics of mycobacteriophage D28 and D29. Can J. Microbiol 8:389-399.
28. World Health Organization. 2010. Global tuberculosis control: WHO report 2010. World Health Organization, Geneva.
29. World Health Organization. Stop TB Dept. 2008. Policy guidance on drug-susceptibility testing (DST) of second-line antituberculosis drugs. World Health Organization, Geneva.
30. Real-Time PCR Using Mycobacteriophage DNA for Rapid Phenotypic Drug Susceptibility Results for Mycobacterium tuberculosis. Pholwat et al., J. Clin. Microbiol. March 2012 vol. 50 no. 3 754-76
31. Rapid First- and Second-Line Drug Susceptibility Assay for Mycobacterium tuberculosis Isolates by Use of Quantitative PCR, Pholwat et al., Journal of Clinical Microbiology J. Clin. Microbiol.]. Vol. 49, no. 1, pp. 69-75. 2011.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

What is claimed is:

1. A method for determining susceptibility of Mycobacterium tuberculosis to one or more drugs, said method comprising testing a sample comprising Mycobacterium tuberculosis by:
preparing at least one culture from said test sample;
contacting one of said at least one cultures with a drug and optionally contacting said additional cultures of said test sample with additional drugs, wherein each additional culture receives a different drug than the other cultures;
contacting said culture or additional cultures with a mycobacteriophage;
determining the amount of mycobacteriophage present after contacting said culture or additional cultures with said mycobacteriophage; and
comparing the amount of mycobacteriophage present in the treated culture or treated additional cultures with the amount of mycobacteriophage present in an otherwise identical untreated culture;
wherein an increase in mycobacteriophage in an untreated culture relative to a culture treated with a drug is an indication that the Mycobacterium tuberculosis present in the sample is susceptible to the treatment drug,
thereby determining susceptibility of Mycobacterium tuberculosis to one or more drugs.

2. The method of claim 1, wherein said culture is a suspension culture.

3. The method of claim 1, wherein the amount of mycobacteriophage present is determined using real-time PCR and a decrease in real-time PCR cycle threshold (C_{T}) value is an indication of an increase in the amount of mycobacteriophage present.

4. The method of claim 1, wherein said cultures are contacted with said drug or additional drugs and said mycobacteriophage simultaneously.

5. The method of claim 4, wherein said cultures are contacted with said drug or additional drugs prior to being contacted with said mycobacteriophage.

6. The method of claim 5, wherein said cultures are contacted with said drug or additional drugs for up to about 72 hours before being contacted with said mycobacteriophage.

7. The method of claim 6, wherein said cultures are contacted with said drug or additional drugs for up to about 48 hours before being contacted with said mycobacteriophage.

8. The method of claim 7, wherein said cultures are contacted with said drug or additional drugs for up to about 24 hours before being contacted with said mycobacteriophage.

9. The method of claim 1, wherein said cultures are in contact with said mycobacteriophage for up to about 72 hours.

10. The method of claim 9, wherein said cultures are in contact with said mycobacteriophage for up to about 48 hours.

11. The method of claim 10, wherein said cultures are in contact with said mycobacteriophage for up to about 24 hours.

12. The method of claim 1, wherein said mycobacteriophage is D29 mycobacteriophage or L5 mycobacteriophage.

13. The method of claim 1, wherein said drug or additional drugs are selected from the group consisting of isoniazid (INH), streptomycin sulfate (SM), rifampin (RMP), pyrazinamide (PZA), ethambutol (EMB), ethionamide (ETA), capreomycin sulfate (CM), amikacin (AK), kanamycin sulfate (KM), levofloxacin, p-aminosalicylic acid (PAS), D-cycloserine (CS), clofazimine (CF), ofloxacin (OFX), moxifloxacin (MFX), linezolid (LZD), and any new investigational antitubercular drug.

14. The method of claim 13, wherein said drug or additional drugs are added at their critical concentrations.

15. The method of claim 13, wherein the critical concentrations in μg/ml are:

| | |
|---|---|
| Isoniazid | 0.1 |
| Rifampin | 1.0 |
| Ethambutol | 5.0 |
| Streptomycin | 1.0-2.0 |
| Amikacin | 1.0 |
| Kanamycin | 1.0-5.0 |
| Capreomycin | 2.5 |
| Ofloxacin | 2.0 |
| Moxifloxacin | 0.25 |
| Ethionamide | 5.0 |
| para-aminosalicylic acid | 2.0 |
| Cycloserine | 30.0 |
| Linezolid | 1.0. |

16. The method of claim 12, wherein said cultures are contacted with said D29 mycobacteriophage at concentrations of about 10² pfu/ml to about 10⁴ pfu/ml.

17. The method of claim 16, wherein said concentration is about 10³ pfu/ml.

18. The method of claim 3, wherein the PCR cycle thresholds are quantified.

19. The method of claim 18, wherein said PCR cycle thresholds are quantified amongst the test sample, the drug-treated sample, and the starting amount of mycobacteriophage.

20. The method of claim 19, wherein the minimal inhibiting concentration of drug or additional drugs useful against the test mycobacterium is estimated.

21. The method of claim 3, wherein Receiver-Operating Characteristic (ROC) analysis is performed using PASW Statistics Software to define a cut-off in the ΔCt values that compares to agar proportion results.

22. The method of claim 21, wherein said cut-offs between the test sample culture and the drug treated sample culture are between about +0.3 and −6.0.

23. The method of claim 21, wherein said cut-offs yield at least about 80% accurate results.

24. The method of claim 21, wherein said cut-offs yield at least about 90% accurate results.

25. The method of claim 24, wherein said cut-offs yield at least about 95% accurate results or about 98% accurate results.

26. The method of claim 1, wherein at least 2 different drugs are tested.

27. The method of claim 26, wherein at least 4 different drugs are tested.

28. The method of claim 27, wherein at least 10 different drugs are tested.

29. The method of claim 28, wherein 13 different drugs are tested.

30. The method of claim 1, wherein the amount of mycobacteriophage present is determined at multiple intervals following contact with said mycobacteriophage.

31. The method of claim 1, wherein said sample is from a subject suspected of having tuberculosis.

32. The method of claim 1, wherein when multiple drugs are tested said drugs are tested simultaneously in a multiwell device or in multiple chambers.

33. The method of claim 32, wherein said multiwell device is a multiwell plate.

34. The method of claim 33, wherein the number of wells in said multiwell plate is selected from the group consisting of 6 well, 12 well, 24 well, 48 well, 96 well, 384 well, and 1536 well plates.

35. The method of claim 1, wherein said susceptibility is determined in less than about 5 days.

36. The method of claim 35, wherein said susceptibility is determined in less than about 4 days.

37. The method of claim 36, wherein said susceptibility is determined in less than about 3 days.

38. The method of claim 37, wherein said susceptibility is determined in less than about 2 days.

39. The method of claim 3, wherein said PCR is performed using primers selected from the group of primers having SEQ ID NOs:1-6, wherein a primer pair comprises one forward and one reverse primer.

40. The method of claim 1, wherein a drug susceptibility profile is determined and said profile is used to select a drug treatment regimen for a subject from whom the mycobacterium tuberculosis was obtained.

41. The method of claim 1, further wherein said sample is tested using the standard agar proportion method and the results of each method are compared.

42. The method of claim 1, further wherein said sample is tested using the Mycobacterium tuberculosis 16S rRNA propidium monoazide assay and the results of each method are compared.

43. The method of claim 1, wherein said test sample is obtained from a subject.

44. The method of claim 43, wherein said sample is purified or cultured to obtained more mycobacteria prior to beginning the drug susceptibility assay.

45. The method of claim 43, wherein said sample is selected from the group consisting of sputum, CSF, blood, blood components, serum, plasma, pleural effusion, gastric aspirates, urine, throat swab, and stools.

46. A method for treating tuberculosis in a subject in need thereof, said method comprising testing a subject suspected of having tuberculosis using the methods of claim 1 to determine the drug susceptibility of the strain of Mycobacterium tuberculosis in said subject, and then treating said subject with said drug or drugs that the strain of Mycobacterium tuberculosis is susceptible to, thereby treating tuberculosis in a subject in need thereof.

47. The method of claim 46, wherein said Mycobacterium tuberculosis is multidrug resistant or extensively drug-resistant.

48. The method of claim 1, wherein the amount of mycobacteriophage present is determined using a reporter dye or an internal probe.

49. A kit for screening susceptibility of Mycobacterium tuberculosis to one or more drugs, said kit comprising optionally one or more of the following: one or more drugs, culture medium, PCR reagents and primers, at least one strain of Mycobacterium tuberculosis for culturing, optionally a mycobacteriophage, and an instructional material for the use thereof.


SEQUENCE LISTING (7 sequences)

SEQ ID NO: 1 - DNA, 20 bp, Mycobacteriophage D29
agccgatcagaagcacgggc

SEQ ID NO: 2 - DNA, 20 bp, Mycobacteriophage D29
agcggctcttaggaggggcc

SEQ ID NO: 3 - DNA, 19 bp, Mycobacteriophage D29
gccaccaggagccacgaac

SEQ ID NO: 4 - DNA, 23 bp, Mycobacteriophage D29
aatagggaaggagtctgcgtttg

SEQ ID NO: 5 - DNA, 19 bp, Mycobacteriophage D29
ccaccaggagccacgaact

SEQ ID NO: 6 - DNA, 22 bp, Mycobacteriophage D29
agtggcgtagatcaccttgaca

SEQ ID NO: 7 - DNA, 16 bp, Mycobacteriophage D29
tatacccccggaatcg